(19) United States
(12) Patent Application Publication
(10) Pub. No.: US 2010/0130450 A1
(43) Pub. Date: May 27, 2010

(54) Methods of Treating Fungal Infections

(75) Inventors: Kim Lewis, Newton, MA (US); Michael David Lafleur, Braintree, MA (US)

Correspondence Address: WILMERHALE/BOSTON, 60 STATE STREET, BOSTON, MA, 02109, US

(73) Assignee: NORTHEASTERN UNIVERSITY, Boston, MA (US)

(21) Appl. No.: 12/598,679

(22) PCT Filed: May 5, 2008
(86) PCT No.: PCT/US08/05762

§ 371 Date: Feb. 12, 2010

Related U.S. Application Data
(60) Provisional application No. 60/927,466, filed on May 3, 2007.

(51) Int. Cl. A61K 31/655; C12Q 1/06; A61K 31/438; A61K 31/44; A61K 31/16; A61P 31/10; A61P 31/04

(52) U.S. Cl. 514/150; 435/39; 514/280; 514/354; 514/615

(57) ABSTRACT

Methods of identifying compounds that potentiate the activity of antifungal agents, potentiators identified by these methods, and methods of using potentiators are disclosed.

### CROSS REFERENCE TO RELATED APPLICATIONS

This application claims priority from U.S. Provisional Application Ser. No. 60/927,466, filed on May 3, 2007, which is incorporated herein by reference in its entirety.

### FIELD OF THE INVENTION

The invention relates to medicine, and more particularly to the treatment of fungal infections.

### BACKGROUND OF THE INVENTION

Multidrug tolerance of pathogens is in large part the result of the entry of microbial cells into a dormant state. Such dormant cells can be responsible for latent (chronic) diseases or relapsing disorders. Many such dormant cells can be suppressed by known antifungals but have not been eradicated.

Fungal biofilms are communities of cells that settle and proliferate on surfaces and are covered by an exopolymer matrix. They are slow-growing and many are in the stationary phase of growth. They can be formed by most, if not all, pathogens. According to the CDC, 65% of all infections in the United States are caused by biofilms that can be formed by common pathogens. The biofilm exopolymer matrix protects against immune cells, and persister cells that are contained in the biofilm can survive both the onslaught of antifungal treatment and the immune system. When antifungal levels decrease, these persister cells can repopulate the biofilm, which will shed off new planktonic cells, producing a relapsing biofilm infection. Fungal biofilm infections are highly recalcitrant to antifungal treatment. Therefore, there is a need for adequate therapy against these infections.

### SUMMARY OF THE INVENTION

Aspects of the invention are based, in part, upon a new strategy for screening compounds for their ability to potentiate the activity of antifungal agents, e.g., miconazole. Accordingly, in one aspect, the invention features a method of identifying a compound that potentiates the activity of an antifungal agent. The method includes contacting a fungus with an antifungal agent; detecting the number of viable fungal cells in the presence of the antifungal agent; contacting the fungus with a candidate potentiator compound; detecting the number of viable fungal cells in the presence of the candidate potentiator compound; and comparing the numbers of viable fungal cells in the absence and presence of the candidate potentiator compound. By this method, a greater number of viable fungal cells in the absence of the candidate potentiator compound than in the presence of the candidate potentiator compound is indicative that the candidate potentiator compound is a compound that potentiates the activity of an antifungal agent. In some embodiments, the method further comprises identifying such a candidate potentiator compound as a potentiator.

In some embodiments, the method further comprises contacting a second fungus with the candidate potentiator compound in the absence of the antifungal agent, and determining the number of viable fungal cells in the absence and presence of the candidate potentiator compound, wherein the fungus and the second fungus are the same. In certain embodiments, the number of viable fungal cells of the second fungus is substantially similar in the presence and absence of the candidate potentiator compound, i.e., the candidate potentiator compound is not an antifungal agent.

In certain embodiments, the fungus is one or more of the following: a member of the genus Aspergillus (e.g., Aspergillus flavus, Aspergillus fumigatus, Aspergillus glaucus, Aspergillus nidulans, Aspergillus niger, and Aspergillus terreus); Blastomyces dermatitides; a member of the genus Candida (e.g., Candida albicans, Candida glabrata, Candida tropicalis, Candida parapsilosis, Candida krusei, and Candida guillermondii); Coccidioides immitis; a member of the genus Cryptococcus (e.g., Cryptococcus neoformans, Cryptococcus albidus and Cryptococcus laurentii); Histoplasma capsulatum var. capsulatum; Histoplasma capsulatum var. duboisii; Paracoccidioides brasiliensis; Sporothrix schenckii; Absidia corymbifera; Rhizomucor pusillus; and Rhizopus arrhizus. In some embodiments, the fungus is a fungal biofilm. In other embodiments, the fungus comprises persister cells.

In particular embodiments, the number of viable fungal cells is determined in a liquid growth medium. In other embodiments, the number of viable fungal cells is determined in a plate assay.

In yet further embodiments, the candidate potentiator compound is one found in a chemical library, such as the Compound Library of The New England Regional Center of Excellence for Biodefense and Emerging Infectious Diseases, the Compound Library of the National Institutes of Health Molecular Library Screening Center, The ChemBridge Library, the ChemDiv Library, and the MayBridge Library.

In still another aspect, the invention features potentiators identified by any of the methods described herein. In another aspect, the invention features methods of inhibiting the growth of, or killing, a fungus by contacting the fungus with an effective amount of an antifungal agent in combination with an effective amount of a potentiator identified by any of the methods described herein. In yet another aspect, the invention features pharmaceutical formulations that contain a potentiator identified by any of the methods described herein in combination with a pharmaceutically acceptable carrier. In still other aspects, the invention features methods of treating a fungal infection in a subject in need thereof comprising administering to the subject an effective amount of an antifungal agent in combination with an effective amount of a potentiator identified by any of the methods described herein.

In one aspect, the invention features potentiator compounds of the Formula I:

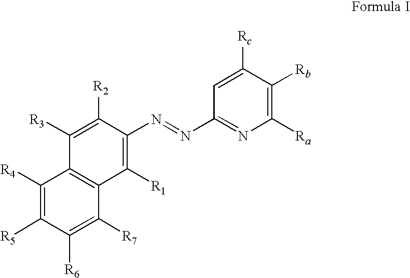

and pharmaceutically acceptable salts, hydrates, and solvates thereof, wherein

R₁-R₇ and Rₐ-R_{c} are each independently —H, halogen, amino, alkylamino, nitro, hydroxyl, cyano, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃ alkyl, —NHC(O)—C₁-C₆ alkyl, aryl, arylalkyl, heteroaryl, heteroarylalkyl,

with the proviso that when R₁ is OH, R_{b} is not H or Cl.

In another aspect, the invention features potentiator compounds of Formula II:

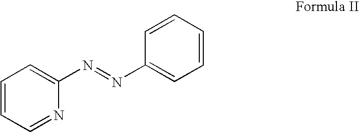

and pharmaceutically acceptable salts, hydrates, and solvates thereof,

wherein any one or more —H can be independently substituted with any one of the following substituents: halogen; —NO₂; —NH₂; alkylamino, hydroxyl; cyano; C₁₋₆ alkyl; C₂₋₆ alkenyl; C₂₋₆ alkynyl; C₁₋₆ alkoxy; —C(O)C₁₋₆alkyl; —C(O)C₁₋₆alkyl; C₃₋₆cycloalkyl; C₃₋₆cycloalkyl-C₁₋₃alkyl; alkylaryl; aryl; arylalkyl; heteroaryl; or heteroarylalkyl; and

wherein the compound is not (E)-4-(pyridin-2-yldiazenyl)benzene-1,3-diol.

In another aspect, the invention features potentiator compounds of Formula III:

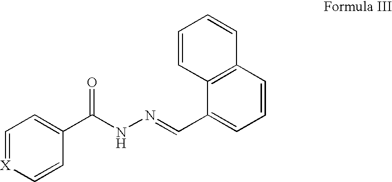

and pharmaceutically acceptable salts, hydrates, and solvates thereof,

wherein any one or more —H can be independently substituted with any one of the following substituents: halogen; —NO₂; —NH₂; alkylamino, hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl;

wherein X is N or C(H); and

wherein the compound is not (E)-N′-((2-hydroxynaphthalen-1-yl)methylene)benzohydrazide or (E)-N′-((2-hydroxynaphthalen-1-yl)methylene)isonicotinohydrazide.

In another aspect, the invention features potentiator compounds of Formula IV:

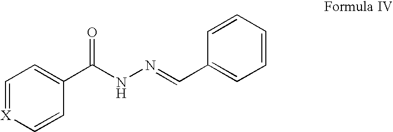

and pharmaceutically acceptable salts, hydrates, and solvates thereof,

wherein any one or more —H can be independently substituted with any one of the following substituents: halogen; —NO₂; —NH₂; alkylamino, hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl;

wherein X is N or C(H); and

wherein the compound is not (E)-N′-(2-hydroxybenzylidene)-4-nitrobenzohydrazide, (E)-N′-(2-hydroxybenzylidene)isonicotinohydrazide, or (E)-4-bromo-N′-(2-hydroxybenzylidene)benzohydrazide.

In another aspect, the invention features a method of inhibiting the growth of, or killing, a fungus, the method comprising contacting the fungus with (i) an antifungal agent, and (ii) one or more potentiator compounds of Formulae I, II, III, and IV:

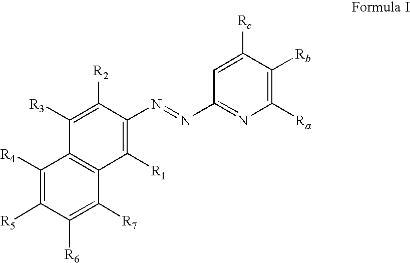

or pharmaceutically acceptable salts, hydrates, and solvates thereof,

wherein R₁-R₇ and Rₐ-R_{c} are each independently —H, halogen, amino, alkylamino, nitro, hydroxyl, cyano, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃ alkyl, —NHC(O)—C₁-C₆ alkyl, aryl, arylalkyl, heteroaryl, heteroarylalkyl;

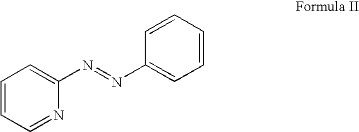

or pharmaceutically acceptable salts, hydrates, and solvates thereof,

wherein any one or more —H can be independently substituted with any one of the following substituents: halogen; —NO₂; —NH₂; alkylamino, hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl;

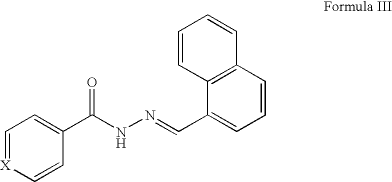

or pharmaceutically acceptable salts, hydrates, and solvates thereof,

wherein any one or more —H can be independently substituted with any one of the following substituents: halogen; —NO₂; —NH₂; alkylamino, hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; and

wherein X is N or C(H); and

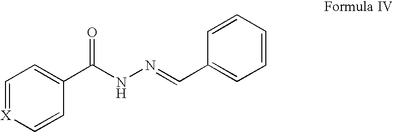

or pharmaceutically acceptable salts, hydrates, and solvates thereof,

wherein any one or more —H can be independently substituted with any one of the following substituents: halogen; —NO₂; —NH₂; alkylamino, hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; and

wherein X is N or C(H);

thereby inhibiting the growth of, or killing, the fungus.

In certain embodiments, the potentiator compound is a potentiator compound having Formula I. In other embodiments, the potentiator compound is a potentiator compound having Formula II. In yet other embodiments, the potentiator compound is a potentiator compound having Formula III. In yet other embodiments, the potentiator compound is a potentiator compound having Formula IV.

In some embodiments, the potentiator compound potentiates the activity of the antifungal agent. In some embodiments, the potentiator compound is not an antifungal compound.

In certain embodiments, the fungus is one or more of the following: a member of the genus Aspergillus (e.g., Aspergillus flavus, Aspergillus fumigatus, Aspergillus glaucus, Aspergillus nidulans, Aspergillus niger, and Aspergillus terreus); Blastomyces dermatitidis; a member of the genus Candida (e.g., Candida albicans, Candida glabrata, Candida tropicalis, Candida parapsilosis, Candida krusei, and Candida guillermondii); Coccidioides immitis; a member of the genus Cryptococcus (e.g., Cryptococcus neoformans, Cryptococcus albidus, and Cryptococcus laurentii); Histoplasma capsulatum var. capsulatum; Histoplasma capsulatum var. duboisii; Paracoccidioides brasiliensis; Sporothrix schenckii; Absidia corymbifera; Rhizomucor pusillus; and Rhizopus arrhizus.

In some embodiments, the fungus is a recalcitrant fungus. In other embodiments, the fungus is a fungal biofilm. In yet other embodiments, the fungus comprises persister cells.

In certain embodiments, the antifungal agent is Amphotericin B, an imidazole (e.g., miconazole), clotrimazole, fluconazole, itraconazole, ketoconazole, ravuconazole, posaconazole, voriconazole, caspofungin, micafungin, FK463, anidulafungin (LY303366), hydroxystilbamidine, 5-fluorocytosine, flucytosine, iodide, terbinafine, Nystatin, griseofulvin, or ciclopirox.

In another aspect, the invention features a method of treating a fungal infection in a subject in need thereof, the method comprising administering to the subject an effective amount of an antifungal agent in combination with an effective amount of one or more potentiator compounds of Formulae I, II, III, and IV:

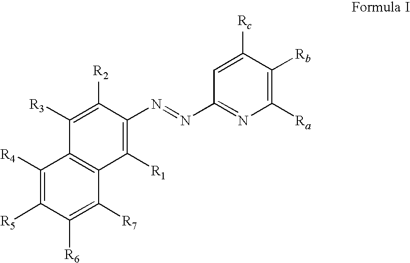

or pharmaceutically acceptable salts, hydrates, and solvates thereof,

wherein R₁-R₇ and Rₐ-R_{c} are each independently —H, halogen, amino, alkylamino, nitro, hydroxyl, cyano, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃ alkyl, —NHC(O)—C₁-C₆ alkyl, aryl, arylalkyl, heteroaryl, heteroarylalkyl;

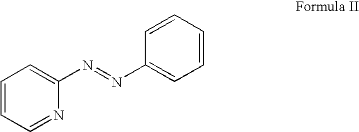

or pharmaceutically acceptable salts, hydrates, and solvates thereof,

wherein any one or more —H can be independently substituted with any one of the following substituents: halogen; —NO₂; —NH₂; alkylamino, hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl;

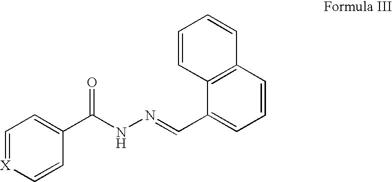

or pharmaceutically acceptable salts, hydrates, and solvates thereof,

wherein any one or more —H can be independently substituted with any one of the following substituents: halogen; —NO₂; —NH₂; alkylamino, hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; and

wherein X is N or C(H); and

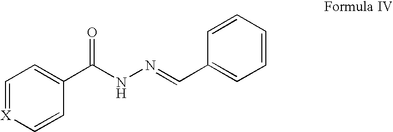

or pharmaceutically acceptable salts, hydrates, and solvates thereof,

wherein any one or more —H can be independently substituted with any one of the following substituents: halogen; —NO₂; —NH₂; alkylamino, hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; and

wherein X is N or C(H);

thereby treating the fungal infection.

In certain embodiments, the potentiator compound is a potentiator compound having Formula I. In other embodiments, the potentiator compound is a potentiator compound having Formula II. In yet other embodiments, the potentiator compound is a potentiator compound having Formula III. In yet other embodiments, the potentiator compound is a potentiator compound having Formula IV.

In some embodiments, the potentiator compound potentiates the activity of the antifungal agent. In some embodiments, the potentiator compound is not an antifungal compound.

In certain embodiments, the fungal infection comprises one or more of the following: a member of the genus Aspergillus (e.g., Aspergillus flavus, Aspergillus fumigatus, Aspergillus glaucus, Aspergillus nidulans, Aspergillus niger, and Aspergillus terreus); Blastomyces dermatitidis; a member of the genus Candida (e.g., Candida albicans, Candida glabrata, Candida tropicalis, Candida parapsilosis, Candida krusei, and Candida guillermondii); Coccidioides immitis; a member of the genus Cryptococcus (e.g., Cryptococcus neoformans, Cryptococcus albidus, and Cryptococcus laurentii); Histoplasma capsulatum var. capsulatum; Histoplasma capsulatum var. duboisii; Paracoccidioides brasiliensis; Sporothrix schenckii; Absidia corymbifera; Rhizomucor pusillus; and Rhizopus arrhizus.

In some embodiments, the fungus is a recalcitrant fungus. In other embodiments, the fungus is a fungal biofilm. In yet other embodiments, the fungus comprises persister cells.

In certain embodiments, the antifungal agent is Amphotericin B, an imidazole (e.g., miconazole), clotrimazole, fluconazole, itraconazole, ketoconazole, ravuconazole, posaconazole, voriconazole, caspofungin, micafungin, FK463, anidulafungin (LY303366), hydroxystilbamidine, 5-fluorocytosine, flucytosine, iodide, terbinafine, Nystatin, griseofulvin, or ciclopirox.

In some embodiments, the fungal infection is aspergillosis, blastomycosis, candidiasis (e.g., oral thrush or vaginitis), coccidioidomycosis, cryptococcosis, histoplasmosis, paracoccidiomycosis, sporotrichosis, or zygomycosis. In some embodiments, the fungal infection is associated with a catheter, an orthopedic prostheses, or a heart valve.

In another aspect, the invention features a method of inhibiting the growth of, or killing, a fungus, the method comprising contacting the fungus with effective amounts of (i) an antifungal agent, and (ii) one or more of potentiator

### Compounds 1-12:

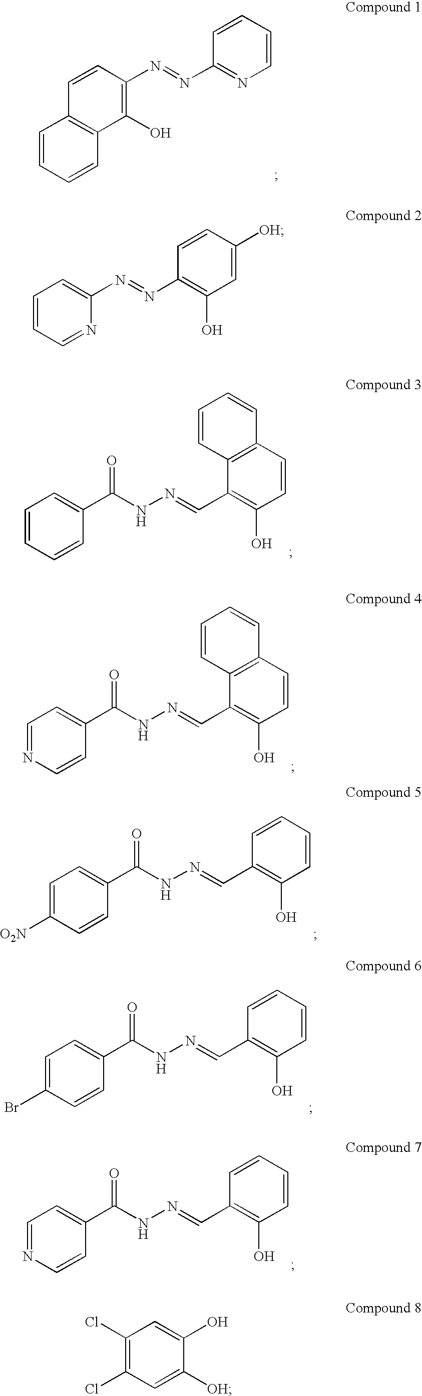

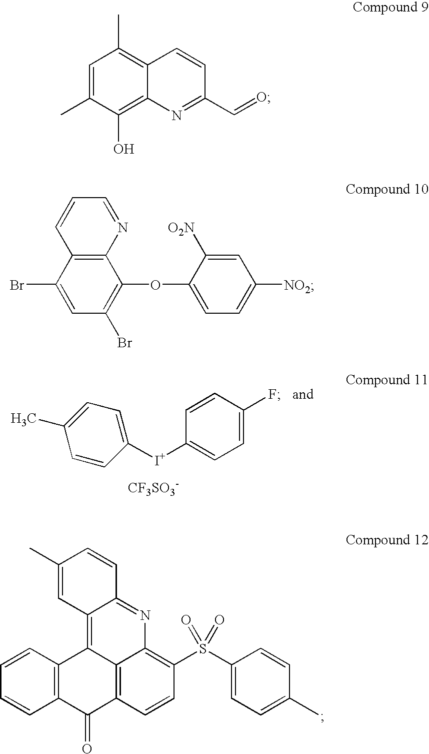

or one or more of a pharmaceutically acceptable salt, hydrate, or solvate of

### Compounds 1-12,

thereby inhibiting the growth of, or killing, the fungus.

In some embodiments, potentiator Compounds 1-12 potentiate the activity of the antifungal agent. In some embodiments, potentiator Compounds 1-12 are not antifungal compounds.

In certain embodiments, the fungus is one or more of the following: a member of the genus Aspergillus (e.g., Aspergillus flavus, Aspergillus fumigatus, Aspergillus glaucus, Aspergillus nidulans, Aspergillus niger, and Aspergillus terreus); Blastomyces dermatitidis; a member of the genus Candida (e.g., Candida albicans, Candida glabrata, Candida tropicalis, Candida parapsilosis, Candida krusei, and Candida guillermondii); Coccidioides immitis; a member of the genus Cryptococcus (e.g., Cryptococcus neoformans, Cryptococcus albidus, and Cryptococcus laurentii); Histoplasma capsulatum var. capsulatum; Histoplasma capsulatum var. duboisii; Paracoccidioides brasiliensis; Sporothrix schenckii; Absidia corymbifera; Rhizomucor pusillus; and Rhizopus arrhizus.

In some embodiments, the fungus is a recalcitrant fungus. In other embodiments, the fungus is a fungal biofilm. In yet other embodiments, the fungus comprises persister cells.

In certain embodiments, the antifungal agent is Amphotericin B, an imidazole (e.g., miconazole), clotrimazole, fluconazole, itraconazole, ketoconazole, ravuconazole, posaconazole, voriconazole, caspofungin, micafungin, FK463, anidulafungin (LY303366), hydroxystilbamidine, 5-fluorocytosine, flucytosine, iodide, terbinafine, Nystatin, griseofulvin, or ciclopirox.

In another aspect, the invention features a method of inhibiting the growth of, or killing, a fungus, the method comprising contacting the fungus with effective amounts of (i) an antifungal agent, and (ii) potentiator Compound 1:

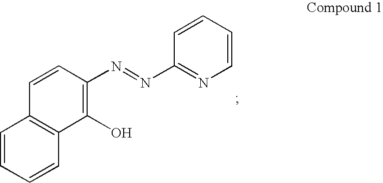

or a pharmaceutically acceptable salt, hydrate, or solvate of potentiator Compound 1,
thereby inhibiting the growth of, or killing, the fungus.

In some embodiments, potentiator Compound 1 potentiates the activity of the antifungal agent. In some embodiments, potentiator Compound 1 is not an antifungal compound.

In certain embodiments, the fungus is one or more of the following: a member of the genus Aspergillus (e.g., Aspergillus flavus, Aspergillus fumigatus, Aspergillus glaucus, Aspergillus nidulans, Aspergillus niger, and Aspergillus terreus); Blastomyces dermatitidis; a member of the genus Candida (e.g., Candida albicans, Candida glabrata, Candida tropicalis, Candida parapsilosis, Candida krusei, and Candida guillermondii); Coccidioides immitis; a member of the genus Cryptococcus (e.g., Cryptococcus neoformans, Cryptococcus albidus, and Cryptococcus laurentii); Histoplasma capsulatum var. capsulatum; Histoplasma capsulatum var. duboisii; Paracoccidioides brasiliensis; Sporothrix schenckii; Absidia corymbifera; Rhizomucor pusillus; and Rhizopus arrhizus.

In some embodiments, the fungus is a recalcitrant fungus. In other embodiments, the fungus is a fungal biofilm. In yet other embodiments, the fungus comprises persister cells.

In certain embodiments, the antifungal agent is Amphotericin B, an imidazole (e.g., miconazole), clotrimazole, fluconazole, itraconazole, ketoconazole, ravuconazole, posaconazole, voriconazole, caspofungin, micafungin, FK463, anidulafungin (LY303366), hydroxystilbamidine, 5-fluorocytosine, flucytosine, iodide, terbinafine, Nystatin, griseofulvin, or ciclopirox.

In another aspect, the invention features a method of treating a fungal infection in a subject in need thereof, the method comprising administering to the subject an effective amount of an antifungal agent in combination with an effective amount of one or more of potentiator Compounds 1-12:

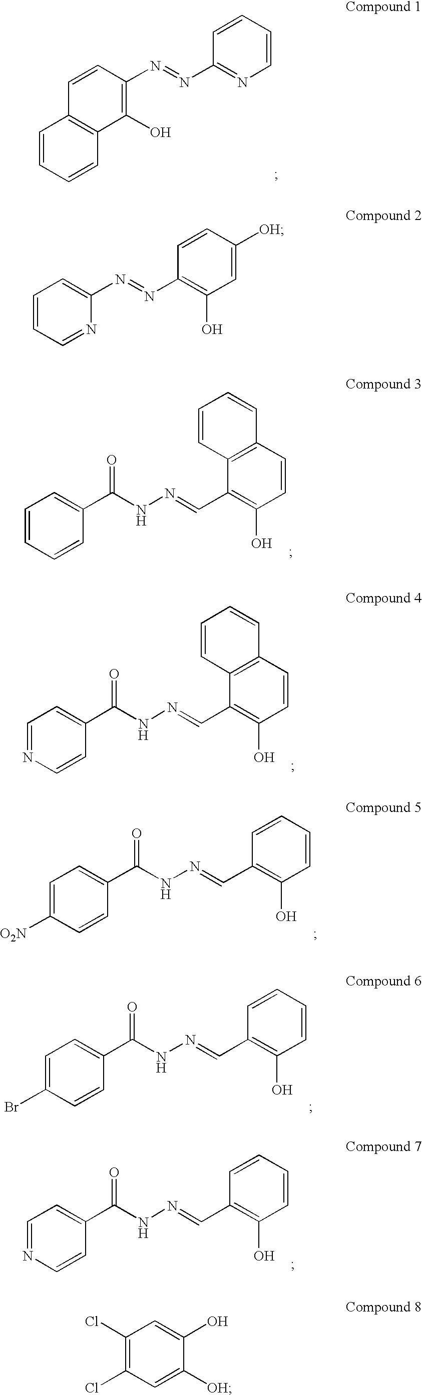

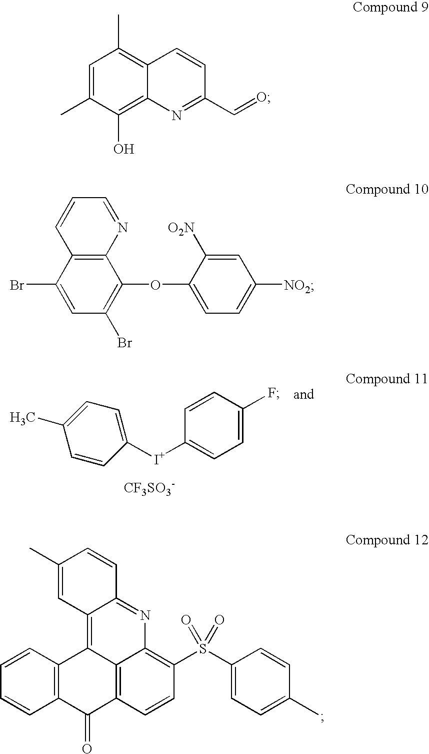

or one or more pharmaceutically acceptable salt, hydrate, or solvate of potentiator Compounds 1-12, thereby treating the fungal infection.

In some embodiments, potentiator Compounds 1-12 potentiate the activity of the antifungal agent. In some embodiments, the potentiator Compounds 1-12 are not antifungal compounds.

In certain embodiments, the fungal infection comprises one or more of the following: a member of the genus Aspergillus (e.g., Aspergillus flavus, Aspergillus fumigatus, Aspergillus glaucus, Aspergillus nidulans, Aspergillus niger, and Aspergillus terreus); Blastomyces dermatitides; a member of the genus Candida (e.g., Candida albicans, Candida glabrata, Candida tropicalis, Candida parapsilosis, Candida krusei, and Candida guillermondii); Coccidioides immitis; a member of the genus Cryptococcus (e.g., Cryptococcus neoformans, Cryptococcus albidus, and Cryptococcus laurentii); Histoplasma capsulatum var. capsulatum; Histoplasma capsulatum var. duboisii; Paracoccidioides brasiliensis; Sporothrix schenckii; Absidia corymbifera; Rhizomucor pusillus; and Rhizopus arrhizus.

In some embodiments, the fungus is a recalcitrant fungus. In other embodiments, the fungus is a fungal biofilm. In yet other embodiments, the fungus comprises persister cells.

In certain embodiments, the antifungal agent is Amphotericin B, an imidazole (e.g., miconazole), clotrimazole, fluconazole, itraconazole, ketoconazole, ravuconazole, posaconazole, voriconazole, caspofungin, micafungin, FK463, anidulafungin (LY303366), hydroxystilbamidine, 5-fluorocytosine, flucytosine, iodide, terbinafine, Nystatin, griseofulvin, or ciclopirox.

In some embodiments, the fungal infection is aspergillosis, blastomycosis, candidiasis (e.g., oral thrush or vaginitis), coccidioidomycosis, cryptococcosis, histoplasmosis, paracoccidiomycosis, sporotrichosis, or zygomycosis. In some embodiments, the fungal infection is associated with a catheter, an orthopedic prostheses, or a heart valve.

In another aspect, the invention features a method of treating a fungal infection in a subject in need thereof, the method comprising administering to the subject an effective amount of an antifungal agent in combination with an effective amount of potentiator Compound 1:

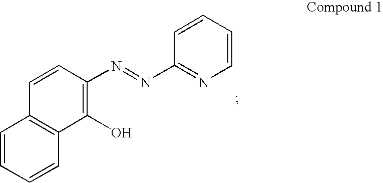

or a pharmaceutically acceptable salt, hydrate, or solvate of Compound 1, thereby treating the fungal infection.

In some embodiments, potentiator Compound 1 potentiates the activity of the antifungal agent. In some embodiments, potentiator Compound 1 is not an antifungal compound.

In certain embodiments, the fungal infection comprises one or more of the following: a member of the genus Aspergillus (e.g., Aspergillus flavus, Aspergillus fumigatus, Aspergillus glaucus, Aspergillus nidulans, Aspergillus niger, and Aspergillus terreus); Blastomyces dermatitidis; a member of the genus Candida (e.g., Candida albicans, Candida glabrata, Candida tropicalis, Candida parapsilosis, Candida krusei, and Candida guillermondii); Coccidioides immitis; a member of the genus Cryptococcus (e.g., Cryptococcus neoformans, Cryptococcus albidus, and Cryptococcus laurentii); Histoplasma capsulatum var. capsulatum; Histoplasma capsulatum var. duboisii; Paracoccidioides brasiliensis; Sporothrix schenckii; Absidia corymbifera; Rhizomucor pusillus; and Rhizopus arrhizus.

In some embodiments, the fungus is a recalcitrant fungus. In other embodiments, the fungus is a fungal biofilm. In yet other embodiments, the fungus comprises persister cells.

In certain embodiments, the antifungal agent is Amphotericin B, an imidazole (e.g., miconazole), clotrimazole, fluconazole, itraconazole, ketoconazole, ravuconazole, posaconazole, voriconazole, caspofungin, micafungin, FK463, anidulafungin (LY303366), hydroxystilbamidine, 5-fluorocytosine, flucytosine, iodide, terbinafine, Nystatin, griseofulvin, or ciclopirox.

In some embodiments, the fungal infection is aspergillosis, blastomycosis, candidiasis (e.g., oral thrush or vaginitis), coccidioidomycosis, cryptococcosis, histoplasmosis, paracoccidiomycosis, sporotrichosis, or zygomycosis. In some embodiments, the fungal infection is associated with a catheter, an orthopedic prostheses, or a heart valve.

In another aspect, the invention features a pharmaceutical composition comprising (i) one or more compounds of Formula (I), Formula (II), Formula (III), and Formula (IV), or pharmaceutically acceptable salts, hydrates, or solvates of compounds of Formula (I), Formula (II), Formula (III), and Formula (IV); and (ii) a pharmaceutically acceptable carrier; wherein Formula (I), Formula (II), Formula (III), and Formula (IV) do not contain the provisos.

In another aspect, the invention features a pharmaceutical composition comprising (i) one or more of Compounds 1-12 described herein, or pharmaceutically acceptable salts, hydrates, or solvates of Compounds 1-12 described herein, and (ii) a pharmaceutically acceptable carrier.

In another aspect, the invention features a method of treating relapsing vaginitis in a subject, the method comprising administering to the subject an effective amount of miconazole in combination with an effective amount of potentiator Compound 1:

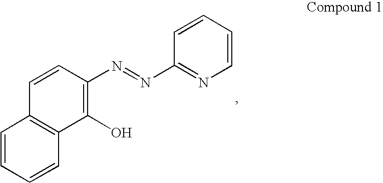

thereby treating the relapsing vaginitis in the subject. In some embodiments, the relapsing vaginitis comprises Candida albicans. In other embodiments, the relapsing vaginitis comprises Candida albicans persister cells.

### DEFINITIONS

“Alkyl” refers to a hydrocarbon chain that may be a straight chain or branched chain, containing the indicated number of carbon atoms. For example, C₁-C₆ indicates that the group may have from 1 to 6 (inclusive) carbon atoms in it.

“Aryl” refers to cyclic aromatic carbon ring systems made from 6 to 18 carbons. Examples of an aryl group include, but are not limited to, phenyl, napthyl, anthracenyl, tetracenyl, and phenanthrenyl. An aryl group can be unsubstituted or substituted with one or more of the following groups: H, OH, ═O, halogen, CN, C₁-C₆ alkyl, C₃-C₆ alkenyl, C₃-C₆ alkynyl, C₁-C₆ alkoxy, C₁-C₃ fluorinated alkyl, NO₂, NH₂, NHC₁-C₆ alkyl, N(C₁-C₆ alkyl)₂, NHC(O)C₁-C₆ alkyl, NHC(O)NHC₁-C₆ alkyl, SO₂NH₂, SO₂NHC₁-C₆ alkyl, SO₂N(C₁-C₆ alkyl)₂, NHSO₂C₁-C₆ alkyl, CO₂C₁-C₆ alkyl, CONHC₁-C₆ alkyl, CON(C₁-C₆ alkyl)₂, or C₁-C₆ alkyl optionally substituted with C₁-C₆ alkyl, C₃-C₆ alkenyl, C₃-C₆ alkynyl, C₁-C₆ alkoxy, CO₂C₁-C₆ alkyl, CN, OH, cycloalkyl, CONH₂, aryl, heteroaryl, COaryl, or trifluoroacetyl.

“Heteroaryl” refers to mono and bicyclic aromatic groups of 4 to 10 atoms containing at least one heteroatom. Heteroatom as used in the term heteroaryl refers to oxygen, sulfur and nitrogen. Examples of monocyclic heteroaryls include, but are not limited to, oxazinyl, thiazinyl, diazinyl, triazinyl, tetrazinyl, imidazolyl, tetrazolyl, isoxazolyl, furanyl, furazanyl, oxazolyl, thiazolyl, thiophenyl, pyrazolyl, triazolyl, and pyrimidinyl. Examples of bicyclic heteroaryls include but are not limited to, benzimidazolyl, indolyl, isoquinolinyl, indazolyl, quinolinyl, quinazolinyl, purinyl, benzisoxazolyl, benzoxazolyl, benzthiazolyl, benzodiazolyl, benzotriazolyl, isoindolyl and indazolyl. A heteroaryl group can be unsubstituted or substituted with one or more of the following groups: H, OH, ═O, halogen, CN, C₁-C₆ alkyl, C₃-C₆ alkenyl, C₃-C₆ alkynyl, C₁-C₆ alkoxy, C₁-C₃ fluorinated alkyl, NO₂, NH₂, NHC₁-C₆ alkyl, N(C₁-C₆ alkyl)₂, NHC(O)C₁-C₆ alkyl, NHC(O)NHC₁-C₆ alkyl, SO₂NH₂, SO₂NHC₁-C₆ alkyl, SO₂N(C₁-C₆ alkyl)₂, NHSO₂C₁-C₆ alkyl, CO₂C₁-C₆ alkyl, CONHC₁-C₆ alkyl, CON(C₁-C₆ alkyl)₂, or C₁-C₆ alkyl optionally substituted with C₁-C₆ alkyl, C₃-C₆ alkenyl, C₃-C₆ alkynyl, C₁-C₆ alkoxy, CO₂C₁-C₆ alkyl, CN, OH, cycloalkyl, CONH₂, aryl, heteroaryl, COaryl, or trifluoroacetyl.

“Arylalkyl” refers to an aryl group with at least one alkyl substitution. Examples of arylalkyl include, but are not limited to, toluenyl, phenylethyl, xylenyl, phenylbutyl, phenylpentyl, and ethylnapthyl. An arylalkyl group can be unsubstituted or substituted with one or more of the following groups: H, OH, ═O, halogen, CN, C₁-C₆ alkyl, C₃-C₆ alkenyl, C₃-C₆ alkynyl, C₁-C₆ alkoxy, C₁-C₃ fluorinated alkyl, NO₂, NH₂, NHC₁-C₆ alkyl, N(C₁-C₆ alkyl)₂, NHC(O)C₁-C₆ alkyl, NHC(O)NHC₁-C₆ alkyl, SO₂NH₂, SO₂NHC₁-C₆ alkyl, SO₂N(C₁-C₆ alkyl)₂, NHSO₂C₁-C₆ alkyl, CO₂C₁-C₆ alkyl, CONHC₁-C₆ alkyl, CON(C₁-C₆ alkyl)₂, or C₁-C₆ alkyl optionally substituted with C₁-C₆ alkyl, C₃-C₆ alkenyl, C₃-C₆ alkynyl, C₁-C₆ alkoxy, CO₂C₁-C₆ alkyl, CN, OH, cycloalkyl, CONH₂, aryl, heteroaryl, COaryl, or trifluoroacetyl.

“Heteroarylalkyl” refers to a heteroaryl group with at least one alkyl substitution. A heteroarylalkyl group can be unsubstituted or substituted with one or more of the following: H, OH, ═O, halogen, CN, C₁-C₆ alkyl, C₃-C₆ alkenyl, C₃-C₆ alkynyl, C₁-C₆ alkoxy, C₁-C₃ fluorinated alkyl, NO₂, NH₂, NHC₁-C₆ alkyl, N(C₁-C₆ alkyl)₂, NHC(O)C₁-C₆ alkyl, NHC(O)NHC₁-C₆ alkyl, SO₂NH₂, SO₂NHC₁-C₆ alkyl, SO₂N(C₁-C₆ alkyl)₂, NHSO₂C₁-C₆ alkyl, CO₂C₁-C₆ alkyl, CONHC₁-C₆ alkyl, CON(C₁-C₆ alkyl)₂, or C₁-C₆ alkyl optionally substituted with C₁-C₆ alkyl, C₃-C₆ alkenyl, C₃-C₆ alkynyl, C₁-C₆ alkoxy, CO₂C₁-C₆ alkyl, CN, OH, cycloalkyl, CONH₂, aryl, heteroaryl, COaryl, or trifluoroacetyl.

“C₁-C₆ alkyl” refers to a straight or branched chain saturated hydrocarbon containing 1-6 carbon atoms. Examples of a C₁-C₆ alkyl group include, but are not limited to, methyl, ethyl, propyl, isopropyl, n-pentyl, isopentyl, neopentyl, and hexyl.

“C₂-C₆ alkenyl” refers to a straight or branched chain unsaturated hydrocarbon containing 2-6 carbon atoms and at least one double bond. Examples of a C₂-C₆ alkenyl group include, but are not limited to, ethylene, propylene, 1-butylene, 2-butylene, isobutylene, sec-butylene, 1-pentene, 2-pentene, isopentene, 1-hexene, 2-hexene, 3-hexene, and isohexene.

“C₃-C₆ alkenyl” refers to a straight or branched chain unsaturated hydrocarbon containing 3-6 carbon atoms and at least one double bond. Examples of a C₃-C₆ alkenyl group include, but are not limited to, propylene, 1-butylene, 2-butylene, isobutylene, sec-butylene, 1-pentene, 2-pentene, isopentene, 1-hexene, 2-hexene, 3-hexene, and isohexene.

“C₂-C₆ alkynyl” refers to a straight or branched chain unsaturated hydrocarbon containing 2-6 carbon atoms and at least one triple bond. Examples of a C₂-C₆ alkynyl group include, but are not limited to, acetylene, propyne, 1-butyne, 2-butyne, isobutyne, sec-butyne, 1-pentyne, 2-pentyne, isopentyne, 1-hexyne, 2-hexyne, and 3-hexyne.

“C₃-C₆ alkynyl” refers to a straight or branched chain unsaturated hydrocarbon containing 3-6 carbon atoms and at least one triple bond. Examples of a C₃-C₆ alkynyl group include, but are not limited to, propyne, 1-butyne, 2-butyne, isobutyne, sec-butyne, 1-pentyne, 2-pentyne, isopentyne, 1-hexyne, 2-hexyne, and 3-hexyne.

“C₁-C₆ alkoxy” refers to a straight or branched chain saturated or unsaturated hydrocarbon containing 1-6 carbon atoms and at least one oxygen atom. Examples of a C₁-C₆ alkoxy include, but are not limited to, methoxy, ethoxy, isopropoxy, butoxy, n-pentoxy, isopentoxy, neopentoxy, and hexoxy.

A “5- to 6-membered monocyclic heterocycle” refers to a monocyclic 5- to 6-membered non-aromatic monocyclic cycloalkyl in which 1-4 of the ring carbon atoms have been independently replaced with a N, O or S atom. When a carbon is replaced by N, the N can be substituted with —H, C₁-C₆ alkyl, or acyl. Representative examples of a 5- to 6-membered monocyclic heterocycle group include, but are not limited to, piperidinyl, piperazinyl, morpholinyl, pyrrolyl, oxazinyl, thiazinyl, diazinyl, triazinyl, tetrazinyl, imidazolyl, tetrazolyl, pyrrolidinyl, isoxazolyl, furanyl, furazanyl, pyridinyl, oxazolyl, thiazolyl, thiophenyl, pyrazolyl, triazolyl, and pyrimidinyl.

Representative “pharmaceutically acceptable salts” include, e.g., water-soluble and water-insoluble salts, such as the acetate, amsonate (4,4-diaminostilbene-2,2-disulfonate), benzenesulfonate, benzoate, bicarbonate, bisulfate, bitartrate, borate, bromide, butyrate, calcium edetate, camsylate, carbonate, chloride, citrate, clavulariate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, and valerate salts.

As used herein, a “potentiator” or a “compound that potentiates” is a compound that supplements or enhances the activity of an antifungal agent, e.g., the antifungal activity of an antifungal agent. In some embodiments, the potentiator is not an antifungal agent, i.e., does not exhibit antifungal activity on its own. In other embodiments, the potentiator is an antifungal agent itself. In some embodiments, the activity of the antifungal agent is synergistic with the activity of the potentiator.

An “effective amount”, when used in connection with a composition described herein, is an amount effective for treating a fungal infection, or for inhibiting the growth of, or killing, a fungus.

As used herein, “about” means a numeric value having a range of ±10% around the cited value.

A “subject”, as used herein, is a mammal, e.g., a human, mouse, rat, guinea pig, dog, cat, horse, cow, pig, or a non-human primate, such as a monkey, chimpanzee, baboon, or rhesus.

As used herein, “treat”, “treating” or “treatment” refers to administering a therapy in an amount, manner (e.g., schedule of administration), and/or mode (e.g., route of administration), effective to improve a disorder (e.g., an infection described herein) or a symptom thereof, or to prevent or slow the progression of a disorder (e.g., an infection described herein) or a symptom thereof. This can be evidenced by, e.g., an improvement in a parameter associated with a disorder or a symptom thereof, e.g., to a statistically significant degree or to a degree detectable to one skilled in the art. An effective amount, manner, or mode can vary depending on the subject and may be tailored to the subject. By preventing or slowing progression of a disorder or a symptom thereof, a treatment can prevent or slow deterioration resulting from a disorder or a symptom thereof in an affected or diagnosed subject.

As used herein, “administered in combination” means that two or more agents are administered to a subject at the same time or within an interval, such that there is overlap of an effect of each agent on the subject. The administrations of the first and second agent can be spaced sufficiently close together such that a combinatorial effect, e.g., a synergistic effect, is achieved. The interval can be an interval of hours, days or weeks. The agents can be concurrently bioavailable, e.g., detectable, in the subject. For example, at least one administration of one of the agents, e.g., an antifungal agent, can be made while the other agent, e.g., a compound described herein, is still present at a therapeutic level in the subject. The subject may have had a response that did not meet a predetermined threshold. For example, the subject may have had a failed or incomplete response, e.g., a failed or incomplete clinical response to the antifungal agent. An antifungal agent and a compound described herein may be formulated for separate administration or may be formulated for administration together.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIG. 1A is a graphic representation of the number of surviving C. albicans 3153A cells after treatment with amphotericin B. FIG. 1B is a graph of the number of surviving C. albicans 3153A cells after treatment with chlorhexidine.

FIG. 2 is a graphic representation of the number of surviving cells following treatment with amphotericin B or chlorhexidine.

FIG. 3 is a graphic representation of the number of surviving cells following treatment with amphotericin B, chlorhexidine, or a combination of amphotericin B and chlorhexidine.

FIG. 4A is a digital representation of a micrograph of live C. albicans planktonic cells.

FIG. 4B is a digital representation of a micrograph of dead C. albicans planktonic cells after treatment with amphotericin B.

FIG. 4C is a digital representation of a micrograph of an untreated C. albicans biofilm.

FIG. 4D is a digital representation of a micrograph of a C. albicans biofilm treated with amphotericin B for 18 hrs.

FIG. 4E is a digital representation of a micrograph of a C. albicans biofilm treated with amphotericin B for 48 hrs.

FIG. 5 is a representation of a microtiter plate containing C. albicans strain CAF4-2 cells and treated with miconazole alone (negative control) or miconazole in combination with various compounds.

FIG. 6 is a digital representation of a microtiter plate containing C. albicans biofilms treated with various concentrations of miconazole and compound AC9.

FIG. 7 is a graphic representation of the number of surviving C. albicans cells from biofilms that were either untreated, treated with miconazole, treated with compound AC9, or treated with a combination of miconazole and AC9.

FIG. 8 is a schematic representation of the chemical structures of verified hits.

### DETAILED DESCRIPTION OF THE INVENTION

This application relates, in part, to novel methods for drug discovery, drugs identified by these methods, and methods of using these drugs. The methods described herein are based on targeted screens for compounds that potentiate the activity of antifungal agents.

### Methods of Identifying Potentiators

The methods described herein are useful for identifying compounds that potentiate the activity of an antifungal agent. The rationale is to screen compounds using fungal strains that are treated with an antifungal agent. The screening methods are readily adapted to high throughput screening (HTS).

In one example, the screen involves contacting a fungus with an antifungal agent. The screen further involves contacting the fungus with a candidate compound. The screen also involves comparing the number of viable cells of the fungus in the presence of the candidate compound to the number of viable cells of the fungus in the absence of the candidate compound. A greater number of viable cells in the absence of the candidate compound compared to the number of viable cells in the presence of the candidate compound is indicative that the candidate compound is a potentiator.

In some situations, the method further includes contacting a second fungus with the candidate compound in the absence of the antifungal agent, and determining the number of viable cells of the second fungus in the absence and presence of the candidate compound, wherein the fungus and the second fungus are the same.

The number of viable cells can be determined by any method known in the art. For example, the fungal cells can be visualized with dyes that discriminate between living and dead cells. Exemplary dyes are XTT, FUN-1, fluorescein diacetate, and those in the LIVE/DEAD® Yeast Viability Kit (Invitrogen). Other nonlimiting examples are described in U.S. Pat. Nos. 5,445,946 and 5,437,980; and Jin et al., Mycopathologia 159:353-360 (2005).

In some instances, the assay is performed on cells grown in a liquid growth medium. In other instances, the number of viable cells is determined in a plate assay, e.g., using cells grown on a microtiter plate.

The screening method can be conducted on any fungus, e.g., one or more of the following: a member of the genus Aspergillus (e.g., Aspergillus flavus, Aspergillus fumigatus, Aspergillus glaucus, Aspergillus nidulans, Aspergillus niger, and Aspergillus terreus); Blastomyces dermatitides; a member of the genus Candida (e.g., Candida albicans, Candida glabrata, Candida tropicalis, Candida parapsilosis, Candida krusei, and Candida guillermondii); Coccidioides immitis; a member of the genus Cryptococcus (e.g., Cryptococcus neoformans, Cryptococcus albidus, and Cryptococcus laurentii); Histoplasma capsulatum var. capsulatum; Histoplasma capsulatum var. duboisii; Paracoccidioides brasiliensis; Sporothrix schenckii; Absidia corymbifera; Rhizomucor pusillus; and Rhizopus arrhizus.

The potentiators identified in the screens can be used to inhibit, reduce, prevent growth of, and/or kill a fungus. Such a fungus can be wherever the fungus grows, including within a subject. Thus, the potentiators can be used to treat a fungal infection in a subject.

In the screens described herein, any candidate compound can be assayed. For example, a candidate compound library can be used to provide a candidate compound. Nonlimiting examples of candidate compound libraries include The Compound Library of the New England Regional Center of Excellence for Biodefense and Emergine Infectious Diseases, The Compound Library of the National Institutes of Health Molecular Library Screening Center, The ChemBridge Library, the ChemDiv Library, and the MayBridge Library. Alternatively, a candidate compound can be synthesized using known methods.

### The Compounds of Formulae I, II, III, and IV

The compositions and methods described herein include compounds according to Formula I:

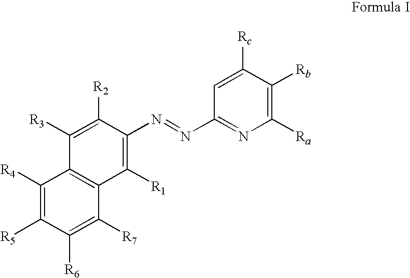

and pharmaceutically acceptable salts, hydrates, and solvates thereof, wherein R₁-R₇ and Rₐ-R_{c} are each independently —H, halogen, amino, alkylamino, nitro, hydroxyl, cyano, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃ alkyl, —NHC(O)—C₁-C₆ alkyl, aryl, arylalkyl, heteroaryl, heteroarylalkyl,
with the proviso that when R₁ is OH, R_{b} is not H or Cl.

The compositions and methods described herein also relate to potentiator compounds of Formula II:

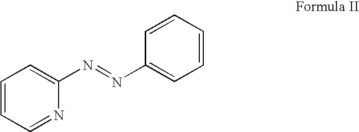

and pharmaceutically acceptable salts, hydrates, and solvates thereof, wherein any one or more —H can be independently substituted with any one of the following substituents: halogen; —NO₂; —NH₂; alkylamino; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; and wherein the compound is not (E)-4-(pyridin-2-yldiazenyl)benzene-1,3-diol.

In addition, the compositions and methods described herein also relate to potentiator compounds of Formula III:

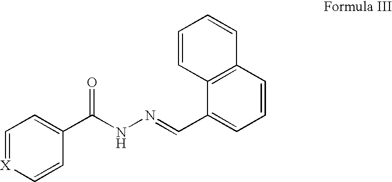

and pharmaceutically acceptable salts, hydrates, and solvates thereof, wherein any one or more —H can be independently substituted with any one of the following substituents: halogen; —NO₂; —NH₂; alkylamino; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; wherein X is N or C(H); and wherein the compound is not (E)-N′-((2-hydroxynaphthalen-1-yl)methylene)benzohydrazide or (E)-N′-((2-hydroxynaphthalen-1-yl)methylene)isonicotinohydrazide.

The compositions and methods described herein also relate to potentiator compounds of Formula IV:

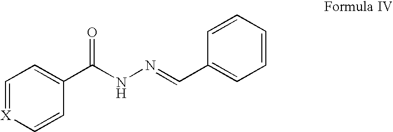

and pharmaceutically acceptable salts, hydrates, and solvates thereof, wherein any one or more —H can be independently substituted with any one of the following substituents: halogen; —NO₂; —NH₂; alkylamino; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; wherein X is N or C(H); and wherein the analog is not (E)-N′-(2-hydroxybenzylidene)-4-nitrobenzohydrazide, (E)-N′-(2-hydroxybenzylidene)isonicotinohydrazide, or (E)-4-bromo-N′-(2-hydroxybenzylidene)benzohydrazide.

The methods and compositions described herein also related to Compounds 1-12 described herein. These compounds are commercially available from ChemBridge Corporation (San Diego, Calif.). Particularly, Compound 1 described herein is available from the ChemBridge DiverSet E library, plate number E-08-89, well G4, and has the ChemBridge identification number 5175171. Compound 1 is also available from the ChemBridge website: https://www.hit21ead.com/.

### Methods of Making Potentiators

If not commercially obtained, the compounds described herein can be synthesized by a variety of methods known to those of skill in the art. One non-limiting example is azo coupling of azides with activated aromatics. As used herein, activated aromatic “means an aromatic ring with a higher electron density resulting from electron donating groups such as —OR or —NR₃. In azo coupling reactions, aromatic diazonium ions are reacted as electrophiles with activated aromatics such as anilines or phenols. The substitution normally occurs at the para position, except when this position is already occupied, in which case ortho position is favored. During the reaction, the pH of solution should be mildly acidic or neutral, since no reaction takes place if the pH is too low.

The synthesis of the compounds described herein can be accomplished according to the following schemes.

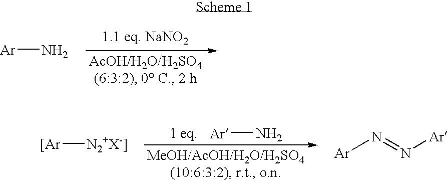

As represented in Scheme 1, aromatic amines can be transformed into the corresponding azide using sodium nitrite in acids such as acetic acid and H₂SO₄. The azide can then be reacted with the same or a different aromatic amine thereby producing the target aryl diazo compound (Wang et al., Dyes and Pigments, 57:77-86 (2003)).

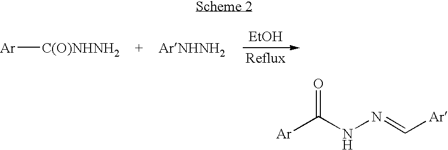

As represented in Scheme 2, arylidenebenzo(or naphtho)hydrazides can be synthesized by reacting an equimolar mixture of an aromaticacylhydrazide, such as 4-nitrosalicylhydrazide, and an arylhydrazine, such as o-hydroxyphenylhydrazine, in ethanol under reflux in a round-bottomed flask for about 3 hr. The resulting precipitate can be collected by filtration and washed with methanol and diethylether (see Lin et al., Acta Cryst. E63, o2864 (2007)).

### Methods of Treating Fungal Infections

The potentiator compounds described herein can be used in combination with known antifungal agents to treat a variety of fungal infections, but have no antifungal activity of their own. Alternatively, certain potentiator compounds have antifungal activity but also act to potentiate the activity of an antifungal agent as well.

### Fungi and Fungal Infections

The fungal infections that can be treated include, but are not limited to, aspergillosis, blastomycosis, candidiasis (e.g., oral thrush or vaginosis), coccidioidomycosis, cryptococcosis, histoplasmosis, paracoccidiomycosis, sporotrichosis, and zygomycosis. Some fungal infections can be associated with indwelling devices, such as catheters and prostheses, and the potentiators described herein can be used to treat them. The potentiator compounds described herein can also be used to treat invasive fungal diseases. In some situations, the potentiator compounds described herein can also be used to treat such infections and diseases in immunodeficient individuals, such as neutropenic individuals undergoing chemotherapy.

Fungal infections are caused by a number of fungal species, each of which can be treated with the compounds and methods described herein. These include, but are not limited to, a member of the genus Aspergillus (e.g., Aspergillus flavus, Aspergillus fumigatus, Aspergillus glaucus, Aspergillus nidulans, Aspergillus niger, and Aspergillus terreus); Blastomyces dermatitidis; a member of the genus Candida (e.g., Candida albicans, Candida glabrata, Candida tropicalis, Candida parapsilosis, Candida krusei, and Candida guillermondii); Coccidioides immitis; a member of the genus Cryptococcus (e.g., Cryptococcus neoformans, Cryptococcus albidus; and Cryptococcus laurentii); Histoplasma capsulatum var. capsulatum; Histoplasma capsulatum var. duboisii; Paracoccidioides brasiliensis; Sporothrix schenckii; Absidia corymbifera; Rhizomucor pusillus; and Rhizopus arrhizus.

### Antifungal Agents

The potentiator compounds described herein can be used in combination with any known antifungal agent. Useful antifungal agents include, but are not limited to, Amphotericin (e.g., Amphotericin B, Amphotericin B Lipid Complex (ABLC), Liposomal Amphotericin B (L-AMB), and Amphotericin B Colloidal Dispersion (ABCD)), azoles (e.g., an imidazole (e.g., miconazole, e.g., Monistat®), clotrimazole, fluconazole, itraconazole, ketoconazole, ravuconazole, posaconazole, and voriconazole), caspofungin, micafungin, FK463, anidulafungin (LY303366), hydroxystilbamidine, 5-fluorocytosine, flucytosine, iodide (e.g., as a saturated solution of potassium iodide, or SSKI), terbinafine, Nystatin, griseofulvin, and ciclopirox. One exemplary antifungal agent is miconazole, e.g., Monistat®, which is an imidazole antifungal agent commonly applied topically to treat fungal infections. These and other antifungal agents are known to those of ordinary skill in the art and are available commercially. For example, many of these antifungal agents are commercially available from Pfizer Inc.; McNeil-PPC, Inc; Johnson & Johnson; Enzon Pharmaceuticals, Inc.; Schering-Plough HealthCare Products; Sandoz Inc.; Ranbaxy Laboratories Ltd.; Mylan Pharmaceuticals, Inc.; Roxane Laboratories, Inc.; Sicor Pharmaceuticals, Inc.; Novopharm Ltd.; Apotex Inc.; Bedford Laboratories; Pliva Inc.; Taro Pharmaceutical Industries, Ltd.; and American Pharmaceutical Partners, Inc.

### Therapeutic Administration

The route and/or mode of administration of an antifungal agent and a potentiator compound described herein can vary depending upon the desired results. For example, the doses of the antifungal agent and a compound described herein can be chosen such that the therapeutic effect is at least 10%, 20%, 25%, 30%, 40%, 50%, 60%, 70%, 80%, 90%, 100%, 125%, 150%, 175%, or 200% greater than that achieved with the antifungal agent alone (i.e., in the absence of a compound described herein). Such effects can be recognized by those skilled in the art, e.g., using standard parameters associated with fungal infections. Dosage regimens can be adjusted to provide the desired response, e.g., a therapeutic response or a combinatorial therapeutic effect. Generally, any combination of doses (either separate or co-formulated) of an antifungal agent and a compound described herein can be used in order to provide a subject with both agents in bioavailable quantities.

Methods of administration include, but are not limited to, intradermal, intramuscular, intraperitoneal, intravenous, subcutaneous, intranasal, epidural, oral, sublingual, intracerebral, intravaginal, transdermal, rectal, by inhalation, or topical, particularly to the ears, nose, eyes, or skin. In some instances, administration can result in release of a potentiator and/or an antifungal agent described herein into the bloodstream. The mode of administration is left to the discretion of the practitioner.

In some instances, a potentiator and/or an antifungal agent described herein can be administered locally. This can be achieved, for example, by local infusion during surgery, topical application (e.g., in a cream or lotion), by injection, by means of a catheter, by means of a suppository or enema, or by means of an implant, said implant being of a porous, non-porous, or gelatinous material, including membranes, such as sialastic membranes, or fibers.

In some situations, a potentiator and/or an antifungal agent described herein can be introduced into the central nervous system, circulatory system or gastrointestinal tract by any suitable route, including intraventricular, intrathecal injection, paraspinal injection, epidural injection, enema, and by injection adjacent to the peripheral nerve. Intraventricular injection can be facilitated by an intraventricular catheter, for example, attached to a reservoir, such as an Ommaya reservoir.

This disclosure also features a device for administering an antifungal agent and a compound described herein. The device can include, e.g., one or more housings for storing pharmaceutical compositions, and can be configured to deliver unit doses of an antifungal agent and a compound described herein. The antifungal agent and a compound described herein can be stored in the same or separate compartments. For example, the device can combine the antifungal agent and the compound prior to administration. It is also possible to use different devices to administer the antifungal agent and a compound described herein.

Pulmonary administration can also be employed, e.g., by use of an inhaler or nebulizer, and formulation with an aerosolizing agent, or via perfusion in a fluorocarbon or synthetic pulmonary surfactant.

In some instances, a potentiator and/or an antifungal agent described herein can be delivered in a vesicle, in particular a liposome (see Langer, Science 249:1527-1533 (1990) and Treat et al., Liposomes in the Therapy of Infectious Disease and Cancer pp. 317-327 and pp. 353-365 (1989)).

In yet other situations, a potentiator and/or an antifungal agent described herein can be delivered in a controlled-release system or sustained-release system (see, e.g., Goodson, in Medical Applications of Controlled Release, vol. 2, pp. 115-138 (1984)). Other controlled or sustained-release systems discussed in the review by Langer, Science 249:1527-1533 (1990) can be used. In one embodiment, a pump can be used (Langer, Science 249:1527-1533 (1990); Sefton, CRC Crit. Ref Biomed. Eng. 14:201 (1987); Buchwald et al., Surgery 88:507 (1980); and Saudek et al., N. Engl. J. Med. 321:574 (1989)). In another embodiment, polymeric materials can be used (see Medical Applications of Controlled Release (Langer and Wise eds., 1974); Controlled Drug Bioavailability, Drug Product Design and Performance (Smolen and Ball eds., 1984); Ranger and Peppas, J. Macromol. Sci. Rev. Macromol. Chem. 2:61 (1983); Levy et al., Science 228:190 (1935); During et al., Ann. Neural. 25:351 (1989); and Howard et al., J. Neurosurg. 71:105 (1989)).

In yet other situations, a controlled- or sustained-release system can be placed in proximity of a target of a potentiator and/or an antifungal agent described herein, e.g., the reproductive organs, reducing the dose to a fraction of the systemic dose.

A potentiator and/or an antifungal agent described herein can be formulated as a pharmaceutical composition that includes a suitable amount of a physiologically acceptable excipient (see, e.g., Remington's Pharmaceutical Sciences pp. 1447-1676 (Alfonso R. Gennaro, ed., 19th ed. 1995)). Such physiologically acceptable excipients can be, e.g., liquids, such as water and oils, including those of petroleum, animal, vegetable, or synthetic origin, such as peanut oil, soybean oil, mineral oil, sesame oil and the like. The physiologically acceptable excipients can be saline, gum acacia, gelatin, starch paste, talc, keratin, colloidal silica, urea and the like. In addition, auxiliary, stabilizing, thickening, lubricating, and coloring agents can be used. In one embodiment, the physiologically acceptable excipients are sterile when administered to an animal. The physiologically acceptable excipient should be stable under the conditions of manufacture and storage and should be preserved against the contaminating action of microorganisms. Water is a particularly useful excipient when a potentiator and/or an antifungal agent described herein is administered intravenously. Saline solutions and aqueous dextrose and glycerol solutions can also be employed as liquid excipients, particularly for injectable solutions. Suitable physiologically acceptable excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Other examples of suitable physiologically acceptable excipients are described in Remington's Pharmaceutical Sciences pp. 1447-1676 (Alfonso R. Gennaro, ed., 19th ed. 1995). The pharmaceutical compositions, if desired, can also contain minor amounts of wetting or emulsifying agents, or pH buffering agents.

Liquid carriers can be used in preparing solutions, suspensions, emulsions, syrups, and elixirs. A potentiator and/or an antifungal agent described herein can be dissolved or suspended in a pharmaceutically acceptable liquid carrier such as water, an organic solvent, a mixture of both, or pharmaceutically acceptable oils or fat. The liquid carrier can contain other suitable pharmaceutical additives including solubilizers, emulsifiers, buffers, preservatives, sweeteners, flavoring agents, suspending agents, thickening agents, colors, viscosity regulators, stabilizers, or osmo-regulators. Suitable examples of liquid carriers for oral and parenteral administration include water (particular containing additives described herein, e.g., cellulose derivatives, including sodium carboxymethyl cellulose solution), alcohols (including monohydric alcohols and polyhydric alcohols, e.g., glycols) and their derivatives, and oils (e.g., fractionated coconut oil and arachis oil). For parenteral administration the carrier can also be an oily ester such as ethyl oleate and isopropyl myristate. The liquid carriers can be in sterile liquid form for administration. The liquid carrier for pressurized compositions can be halogenated hydrocarbon or other pharmaceutically acceptable propellant.

A potentiator and/or an antifungal agent described herein can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the composition is in the form of a capsule.

In some instances, a potentiator and/or an antifungal agent described herein is formulated in accordance with routine procedures as a composition adapted for oral administration to humans. Compositions for oral delivery can be in the form of, e.g., tablets, lozenges, buccal forms, troches, aqueous or oily suspensions or solutions, granules, powders, emulsions, capsules, syrups, or elixirs. Orally administered compositions can contain one or more additional agents, for example, sweetening agents such as fructose, aspartame or saccharin; flavoring agents such as peppermint, oil of wintergreen, or cherry; coloring agents; and preserving agents, to provide a pharmaceutically palatable preparation. In powders, the carrier can be a finely divided solid, which is an admixture with a finely divided antifungal agent and/or compound described herein. In tablets, a potentiator and/or an antifungal agent described herein can be mixed with a carrier having compression properties in suitable proportions and compacted in the shape and size desired. The powders and tablets can contain up to about 99% of a potentiator and/or an antifungal agent described herein.

Capsules can contain mixtures of a potentiator and/or an antifungal agent described herein with inert fillers and/or diluents such as pharmaceutically acceptable starches (e.g., corn, potato, or tapioca starch), sugars, artificial sweetening agents, powdered celluloses (such as crystalline and microcrystalline celluloses), flours, gelatins, gums, etc.

Tablet formulations can be made by conventional compression, wet granulation, or dry granulation methods and utilize pharmaceutically acceptable diluents, binding agents, lubricants, disintegrants, surface modifying agents (including surfactants), suspending or stabilizing agents including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes, and ion exchange resins. Surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colloidal silicon dioxide, phosphates, sodium dodecylsulfate, magnesium aluminum silicate, and triethanolamine.

Moreover, when in a tablet or pill form, a potentiator and/or an antifungal agent described herein can be coated to delay disintegration and absorption in the gastrointestinal tract, thereby providing a sustained action over an extended period of time. Selectively permeable membranes surrounding an osmotically active driving a potentiator and/or an antifungal agent described herein can also be suitable for orally administered compositions. In these latter platforms, fluid from the environment surrounding the capsule can be imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be used. Oral compositions can include standard excipients such as mannitol, lactose, starch, magnesium stearate, sodium saccharin, cellulose, and magnesium carbonate. In some situations, the excipients are of pharmaceutical grade.

In other instances, a potentiator and/or an antifungal agent described herein can be formulated for intravenous administration. Compositions for intravenous administration can comprise a sterile isotonic aqueous buffer. The compositions can also include a solubilizing agent. Compositions for intravenous administration can optionally include a local anesthetic such as lignocaine to lessen pain at the site of the injection. The ingredients can be supplied either separately or mixed together in unit dosage form, for example, as a dry lyophilized powder or water-free concentrate in a hermetically sealed container such as an ampule or sachette indicating the quantity of active agent. Where a potentiator and/or an antifungal agent described herein is administered by infusion, it can be dispensed, for example, with an infusion bottle containing sterile pharmaceutical grade water or saline. Where a potentiator and/or an antifungal agent described herein is administered by injection, an ampule of sterile water for injection or saline can be provided so that the ingredients can be mixed prior to administration.

In other circumstances, a potentiator and/or an antifungal agent described herein can be administered across the surface of the body and the inner linings of the bodily passages, including epithelial and mucosal tissues. Such administrations can be carried out using a potentiator and/or an antifungal agent described herein in lotions, creams, foams, patches, suspensions, solutions, and suppositories (e.g., rectal or vaginal). In some instances, a transdermal patch can be used that contains a potentiator and/or an antifungal agent described herein and a carrier that is inert to the antifungal agent and/or compound described herein, is non-toxic to the skin, and that allows delivery of the agent for systemic absorption into the blood stream via the skin. The carrier can take any number of forms such as creams or ointments, pastes, gels, or occlusive devices. The creams or ointments can be viscous liquid or semisolid emulsions of either the oil-in-water or water-in-oil type. Pastes of absorptive powders dispersed in petroleum or hydrophilic petroleum containing a potentiator and/or an antifungal agent described herein can also be used. A variety of occlusive devices can be used to release a potentiator and/or an antifungal agent described herein into the blood stream, such as a semi-permeable membrane covering a reservoir containing the antifungal agent and/or compound described herein with or without a carrier, or a matrix containing the antifungal agent and/or compound described herein.

A potentiator and/or an antifungal agent described herein can be administered rectally or vaginally in the form of a conventional suppository. Suppository formulations can be made using methods known to those in the art from traditional materials, including cocoa butter, with or without the addition of waxes to alter the suppository's melting point, and glycerin. Water-soluble suppository bases, such as polyethylene glycols of various molecular weights, can also be used.

The amount of a potentiator and/or an antifungal agent described herein that is effective for treating an infection can be determined using standard clinical techniques known to those will skill in the art. In addition, in vitro or in vivo assays can optionally be employed to help identify optimal dosage ranges. The precise dose to be employed can also depend on the route of administration, the condition, the seriousness of the condition being treated, as well as various physical factors related to the individual being treated, and can be decided according to the judgment of a health-care practitioner. For example, the dose of a potentiator and/or an antifungal agent described herein can each range from about 0.001 mg/kg to about 250 mg/kg of body weight per day, from about 1 mg/kg to about 250 mg/kg body weight per day, from about 1 mg/kg to about 50 mg/kg body weight per day, or from about 1 mg/kg to about 20 mg/kg of body weight per day. Equivalent dosages can be administered over various time periods including, but not limited to, about every 2 hrs, about every 6 hrs, about every 8 hrs, about every 12 hrs, about every 24 hrs, about every 36 hrs, about every 48 hrs, about every 72 hrs, about every week, about every two weeks, about every three weeks, about every month, and about every two months. The number and frequency of dosages corresponding to a completed course of therapy can be determined according to the judgment of a health-care practitioner.

In some instances, a pharmaceutical composition described herein is in unit dosage form, e.g., as a tablet, capsule, powder, solution, suspension, emulsion, granule, or suppository. In such form, the pharmaceutical composition can be sub-divided into unit doses containing appropriate quantities of a potentiator and/or an antifungal agent described herein. The unit dosage form can be a packaged pharmaceutical composition, for example, packeted powders, vials, ampoules, pre-filled syringes or sachets containing liquids. The unit dosage form can be, for example, a capsule or tablet itself, or it can be the appropriate number of any such compositions in package form. Such unit dosage form can contain from about 1 mg/kg to about 250 mg/kg, and can be given in a single dose or in two or more divided doses.

The invention is further described in the following examples, which do not limit the scope of the invention described in the claims.

### EXAMPLES

### Example 1

### Characterization of C. albicans Persisters

Both planktonic and biofilm populations were examined for the possible presence of persisters. Several compounds including amphotericin B, chlorhexidine, and caspofungin kill Candida biofilms, and these were tested in dose-dependent experiments. A biphasic killing curve revealing a subpopulation of survivors indicates the presence of persister cells.

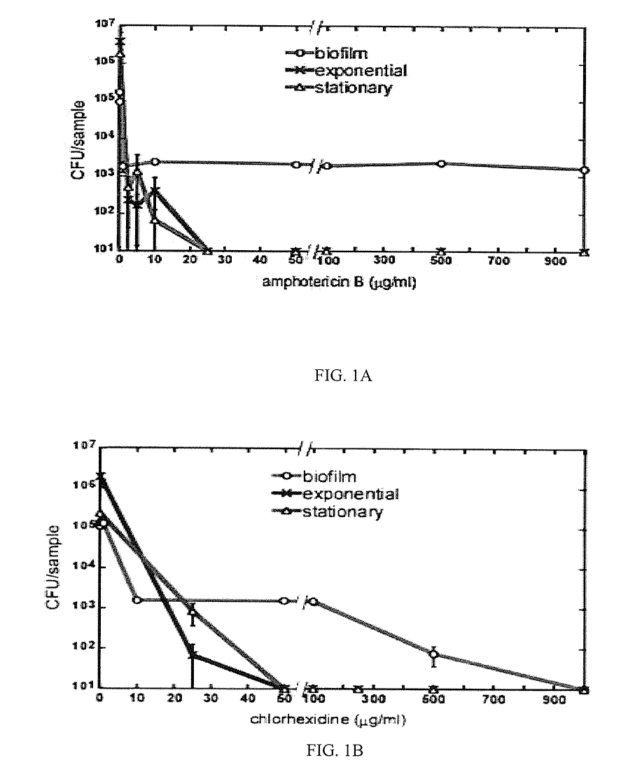

Biofilms of C. albicans 3153A cells were cultured in wells of microtiter plates in RPMI medium for 48 hrs (Ramage et al., Antimicrob. Agents Chemother. 45:2475-2479 (2001)), washed twice in PBS pH 7.4 to remove nonadherent cells, and resuspended in 100 μl RPMI growth medium containing antifungals. After 24 hrs of antifungal challenge, the biofilms and cultures were washed twice, resuspended in 100 μl PBS, scraped, transferred into eppendorf tubes, vortexed and plated for colony forming unit (CFU) determination on YPD medium. Microscopy indicated that the material was a mixture of single cells and clumps of ≦10 cells. This could lead to an underestimation of surviving cells by a factor of ≦10. In parallel, exponentially growing and stationary planktonic cultures were grown for 48 hrs in RPMI medium, and then antifungals were added for 24 hrs. The experiment was performed in triplicate and error bars indicate standard deviation (see FIG. 1).

Caspofungin had a limited effect on biofilms, producing ≦10 fold killing. Amphotericin B effectively killed exponentially growing and stationary cells, with little indication of surviving cells (FIG. 1A). By contrast, a biphasic killing was observed in Candida biofilms, with the majority of the population killed at low concentrations (but above the MIC of 1 μg/ml) while the remaining cells were unaffected by higher concentrations of the drug (FIG. 1A). More than 1% of cells appeared invulnerable to amphotericin B, indicating the presence of persisters in the yeast biofilm, in contrast to observations with bacteria, where stationary planktonic populations produce more persisters than the biofilm. Resistance to killing by amphotericin B, which makes “holes” in the membrane, was unexpected. The activity of this compound depends on, and is limited by, the availability of ergosterol.

Similar to the results seen with amphotericin B, chlorhexidine produced a biphasic killing of the biofilm, while cells in both exponential and stationary cultures were eliminated (FIG. 1B). At higher concentrations (above 100 μg/ml), killing of persisters was observed, and the biofilm was completely sterilized at 1000 μg/ml (a concentration 2-fold lower than what is commonly used in mouthwash and as a therapy for treatment of oral thrush caused by C. albicans (0.2%)).

The biphasic nature of the killing showed that resistant mutants were present in the population. In order to determine whether surviving cells were phenotypic variants of the wild type or whether they were mutants, resistance of the surviving cells was examined.

Biofilms were grown in microtiter plates and were treated with amphotericin B or chlorhexidine (100 μg/ml) for 24 hrs, after which they were washed and vortexed, as discussed above. The cells were then reinoculated into microtiter plates to form new biofilms. The new biofilms, derived from persisters that survived drug treatment, were again treated with the antifungal agents (as discussed above), and the procedure was repeated a total of 3 times. Biofilms were sampled for CFU determination before and after antifungal treatment. The experiment was performed in triplicate.

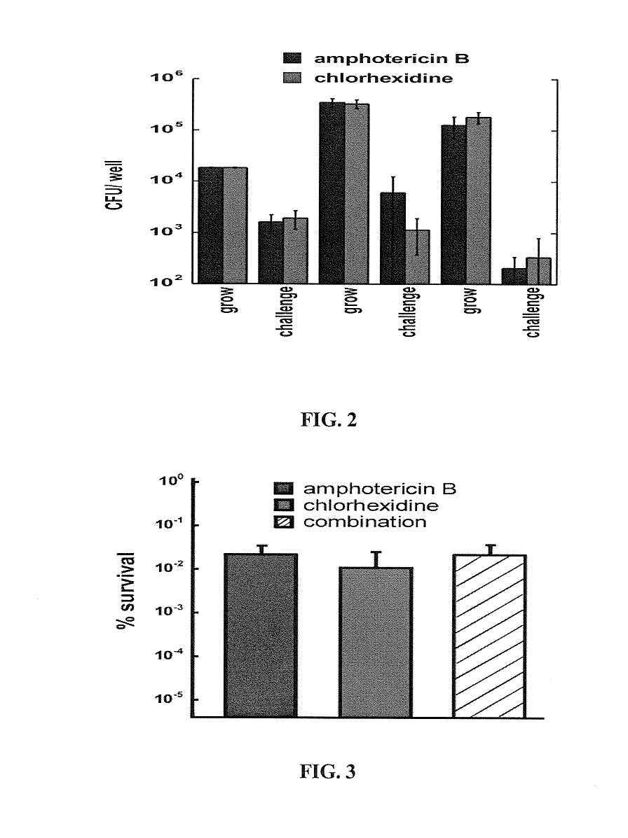

As demonstrated in FIG. 2, the population produced by surviving persisters was not more resistant to drugs, but rather gave rise to a new persister subpopulation (see FIG. 2; error bars indicate standard deviation). If the surviving cells were mutants, complete resistance would be expected upon reapplication of the antifungal or a progressive increase in the numbers of surviving cells with each treatment cycle. Thus, C. albicans persisters were phenotypic variants of the wild type that arose in a clonal population of genetically identical cells.

Tests were also performed to determine if yeast persisters were multidrug tolerant. Mature, 48 hr biofilms of C. albicans were challenged for 24 hrs with 100 μg/ml amphotericin B, 100 μg/ml chlorhexidine, or a combination of these two antifungal agents, using the same procedures discussed above. Biofilms were washed and sampled for CFU determination before and after antifungal treatment, as discussed above.

No additional killing was detected when biofilms were treated with both amphotericin B and chlorhexidine compared to cells treated with individual antifungal agents (FIG. 3; triplicate experiments with error bars indicating standard deviation). Similarly, the number of persisters was essentially the same (1-3%) when biofilms were treated sequentially for 24 hrs with amphotericin B and then chlorhexidine, or vice versa. These experiments indicate the presence of a single uniform persister population.

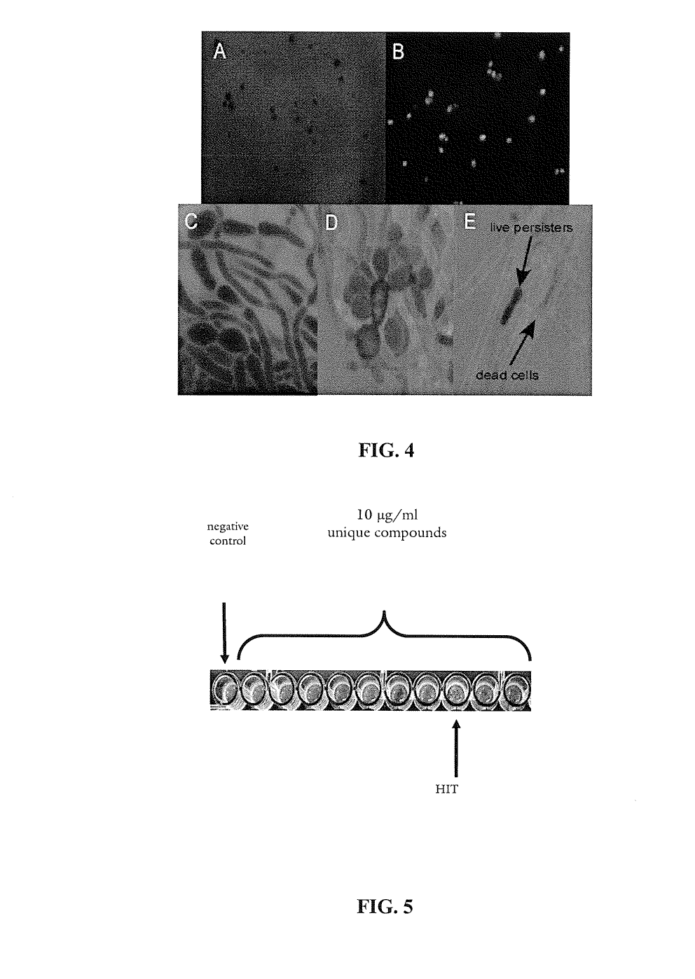

Next, persisters in a biofilm were visualized using several dyes, including fluorescein diacetate, which discriminate between live and dead fungal cells. Planktonic or biofilm cells were stained with 100 μg/ml fluorescein diacetate and examined by fluorescent microscopy. FIG. 4A depicts live planktonic cells; FIG. 4B depicts dead planktonic cells after treatment with 100 μg/ml amphotericin B (400× magnification); FIGS. 4C, D, and E, depict biofilms (1000× magnification) of untreated control, after 18 hrs or after, 48 hrs of amphotericin B treatment (100 μg/ml), respectively.

Exponentially growing C. albicans cells killed with amphotericin B were readily stained with fluorescein diacetate as expected (FIG. 4 A, B). A biofilm was then stained with fluorescein diacetate (FIG. 4C-E). After the addition of amphotericin B, there was a visible decrease in the number of live (dark) cells, and their morphology became aberrant (FIG. 4D). After 48 hrs of amphotericin B treatment, there were only a small number of unstained cells. They appeared as regular pseudohyphae or yeasts and were indistinguishable from morphologically normal untreated cells. Using fluorescence detection and forward scatter, dim persister cells were physically sorted from a disrupted biofilm and grown on agar medium. The sorted cells produced colonies on agar medium, confirming that they were alive. The ability to sort persisters is used to obtain their transcription profile using standard methods.

Given that persisters appeared only in the biofilm, their formation was dependent on the same genes/pathways that determine biofilm development. A large panel of biofilm-defective mutants was therefore tested for their ability to produce persisters by measuring survival to high levels of amphotericin B. These mutants were able to adhere to the surface of a microtiter plate, making it possible to assay in the biofilm survival protocol described above. Unexpectedly, all strains appeared to produce essentially normal levels of persisters (Table 1). This suggests that adherence, rather than subsequent biofilm formation, is the trigger for persister formation.

TABLE 1
Persister formation by biofilm-deficient strains of C. albicans.

| Strain | Genotype | Biofilm architecture | Persisters |
|---|---|---|---|
| 3153A | Control, wild type laboratory strain | Robust 3D wild type | +++¹ |
| CKY357 | CAI-4 mkc1Δ::hisG/mkc1Δ::hisG | Reduced filamentation | ++² |
| | mkc1::pCK70 (URA3) | | |
| CAI4 | SC5314 Δura3::λimm434/Δura3::λimm434 | Robust 3D wild type | +³ |
| CKY136 | CAI-4 efg1::hisG/efg1::hisG ade2::pDBI52 | Filamentation defect; | +++ |
| | (URA3) | sparse monolayer of cells | |
| CKY138 | CAI-4 efg1::hisG/efg1::hisG | Filamentation defect; | ++ |
| | cph1Δ::hisG/cph1Δ::hisG ade2::pDBI52 | sparse monolayer of cells | |
| | (URA3) | | |
| MC191 | ura3Δ::λimm434/ura3Δ::λimm434 | Functionally defective | +++ |
| | arg4::hisG/arg4::hisG his1::hisG/his1::hisG | hyphae | |
| | flo8::ARG4/flo8::HIS1 ade2::URA3/ADE2 | | |
| MC195 | ura3Δ::λimm434/ura3Δ::λimm434 | Robust 3D wild type | + |
| | arg4::hisG/arg4::hisG his1::hisG/his1::hisG | | |
| | flo8::ARG4/flo8::HIS1 ade2::URA3:FLO8- | | |
| | 2/ADE2 | | |
| MC245 | ura3Δ::λimm434/ura3Δ::λimm434 | Robust 3D wild type | ++ |
| | arg4::hisG/arg4::hisG his1::hisG/his1::hisG | | |
| | flo8::ARG4/FLO8 ade2::URA3/ADE2 | | |
| | HIS::his/his | | |
| DAY185 | Δura3::λimm434/Δura3::λimm434 | Robust 3D wild type | ++ |
| | arg4::hisG/arg4::hisG/pARG4-URA3 | | |
| | his1::hisG/his1::hisG/pHIS1 | | |
| DAY286 | Δura3::λimm434/Δura3::λimm434 | Robust 3D wild type | ++ |
| | arg4::hisG/arg4::hisG/pARG4-URA3 | | |
| | his1::hisG/his1::hisG | | |
| GKO443 | Δura3::λimm434/Δura3::λimm434arg4::hisG/ | Biofilm defect; decreased | ++ |
| | arg4::hisG his1::hisG/his1::hisG | biomass | |
| | suv3::Tn7-UAU1/suv3::Tn7-URA3 | | |
| GKO798 | Δura3::λimm434/Δura3::λimm434arg4::hisG/ | Biofilm defect; decreased | ++ |
| | arg4::hisG his1::hisG/his1::hisG | biomass | |
| | kem1::Tn7-UAU1/kem1::Tn7-URA3 | | |
| GKO814 | Δura3::λimm434/Δura3::λimm434arg4::hisG/ | Biofilm defect; decreased | ++ |
| | arg4::hisG his1::hisG/his1::hisG | biomass | |
| | nup85::Tn7-UAU1/nup85::Tn7-URA3 | | |
| GKO9 | Δura3::λimm434/Δura3::λimm434arg4::hisG/ | Biofilm defect; decreased | ++ |
| | arg4::hisG his1::hisG/his1::hisG | biomass | |
| | mds3::Tn7-UAU1/mds3::Tn7-URA3 | | |
| CJN702 | Δura3::λimm434/Δura3::λimm434arg4::hisG/ | Functionally defective | ++ |
| | arg4::hisG his1::hisG::pHIS1/his1::hisG | hyphae | |
| | bcr1::ARG4/bcr1::URA3 | | |
| CJN698 | Δura3::λimm434/Δura3::λimm434arg4::hisG/ | Robust 3D wild type | ++ |
| | arg4::hisG his1::hisG::pHIS1- | | |
| | BCR1/his1::hisG bcr1::ARG4/bcr1::URA3 | | |

¹+++ 1-2% survival,
¹++ 0.1-1% survival,
¹+ 0.05-0.1% survival
²++ indicates 0.1-1% survival
³+ indicates 0.05-0.1% survival

### Example 2

### Screening for Miconazole Potentiators

Given that known antifungals are inactive against persisters (with the exception of high levels of chlorhexidine), a screen was developed to identify potential persister compounds that in combination with a conventional antifungal agent would disable persister formation and eradicate infection. Specifically, a screen was developed using C. albicans cells treated with miconazole at subinhibitory concentrations, to which candidate potentiator compounds were added. This primary screen did not discriminate between directly acting compounds and those that potentiate miconazole. Subsequent validation of primary hits by a checkerboard assay described below allowed identification of synergistically-acting compounds. In order to identify compounds that were active against all forms of cells including biofilms and persisters, the screen was developed against a biofilm population. In order to make the screen compatible with high-throughput approaches, biofilms were grown in microtiter plates and the reduction of a vital dye XTT, commonly used to monitor yeast viability, was used as the quantitative readout.

Microtiter plate wells were seeded with wild type Candida albicans strain CAF4-2. Biofilms were grown for 48 hrs at 37° C., washed with PBS pH 7.4, and resuspended in a final volume of 100 μl RPMI containing 10 μg/ml miconazole. Individual compounds from ChemBridge DiverSet E (ChemBridge Corp., San Diego, Calif.) were then added at 10 μg/ml to the wells. Plates were incubated for 24 hrs and XTT (Sigma-X4751) was added at 1 mg/ml to each well. After 4 hrs of incubation at 37° C., reduction of XTT was monitored by recording the optical density at 690 nm (subtracted from OD₄₅₀) with a microtiter plate reader. Hits were identified when XTT reduction was inhibited by more than 30% of the control values.

Wild type C. albicans with miconazole alone at 10 μg/ml provided a negative control, which showed XTT reduction, indistinguishable from biofilms with no miconazole (see FIG. 5). This concentration was over 300-fold higher than the MIC of logarithmically growing cells (0.03 μg/ml), yet was completely innocuous to the biofilm. Miconazole was added to test wells at 10 μg/ml, and then compounds from the ChemBridge library were added to the same wells at a concentration of 10 μg/ml. After 4 hrs of incubation, the plates were transferred to a microtiter plate reader to quantify and the amount of XTT reduction (FIG. 5). The pilot screen of 5,000 compounds produced 32 hits, with a hit rate of 0.64%. Upon retesting, 13 of the 32 were confirmed, giving a rate of verified hits of 0.26% (false positive rate 0.38%).

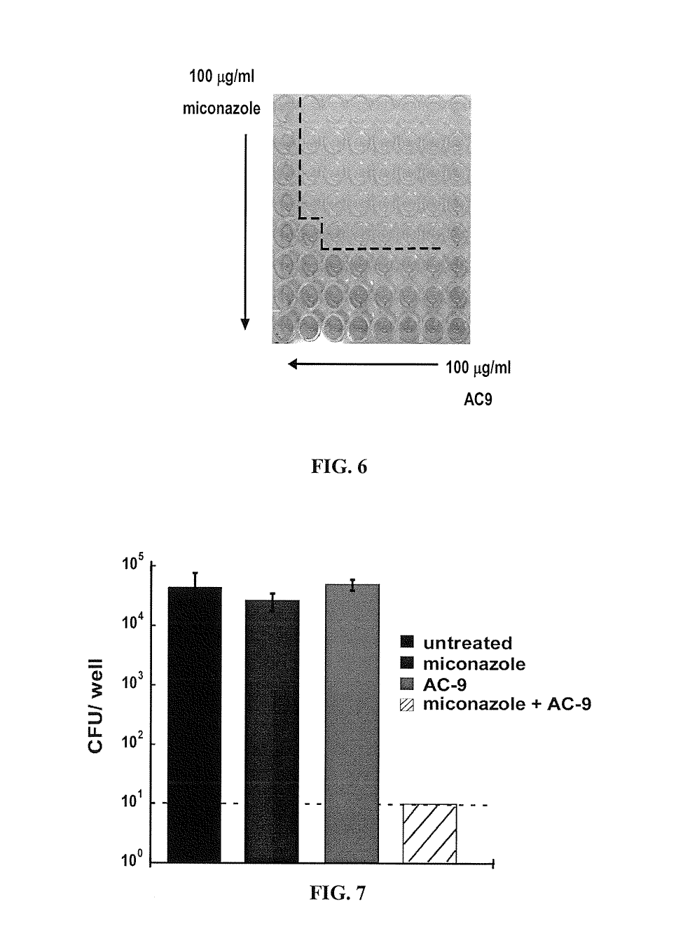

A checkerboard assay was then performed to examine possible synergy between miconazole and the hits. An example of a C. albicans biofilm checkerboard assay with miconazole and compound AC9 is given in FIG. 6. 100 μg/ml miconazole (2× dilution in each subsequent well, y-axis) and 100 μg/ml compound AC9 (2× dilution, x-axis) were added to mature 48 hr wild type C. albicans biofilms and incubated for an additional 24 hrs in the presence of the drugs. XTT was added to each well at 1 mg/ml and biofilms were incubated at 37° C. for an additional 4 hrs. In FIG. 6, biofilms in wells above and to the right of the dashed line (appearing clear) have OD450-OD690 values less than 0.240 and are metabolically inhibited, compared to wells (darker) outside of the dashed line with OD450-OD690>0.240. Note that compound AC-9 itself is red and accounted for some of the dark color seen at the lowest dilutions along the y-axis. A clear synergy was observed and the inhibition of metabolic activity as seen using the XTT assay was evident with AC9 at concentrations as low as 3.1 μg/ml in the presence of 12.5 μg/ml miconazole.

AC9 was found in plate number E-08-89, well G4, and has the ChemBridge identification number 5175171.

Neither miconazole nor AC9 alone inhibited metabolic activity at any concentration tested. The fact that only 1 out of 13 hits had direct activity indicated the high selectivity of the screen towards potentiators. The primary screen was performed at a low concentration of test potentiator compounds (10 μg/ml). This, together with the high level of biofilm resistance, strongly decreased the probability of finding compounds with direct activity.

Next, the same checkerboard assay was repeated to test the ability of the hit compounds to potentiate biofilm killing by miconazole. Mature biofilms of wild type C. albicans were challenged with 100 μg/ml miconazole, 200 μg/ml of the hit compound, or a combination of the two for 24 hrs. Biofilms were then washed, scraped, resuspended in PBS, vortexed for 30 seconds and plated for CFU analysis.

The single compound that showed direct activity had no effect on killing of the biofilm cells either alone or in the presence of miconazole. One of the 12 compounds, AC9, showed killing in the presence of miconazole. Complete eradication of the C. albicans biofilm was observed in the presence of AC9 and 100-200 μg/ml miconazole (FIG. 7; the dashed line indicates the limit of detection). Neither miconazole nor AC9 alone had any killing activity against the biofilm. This result demonstrates the feasibility of developing a synergistic therapy capable of eradicating, rather than merely suppressing, biofilm infections.

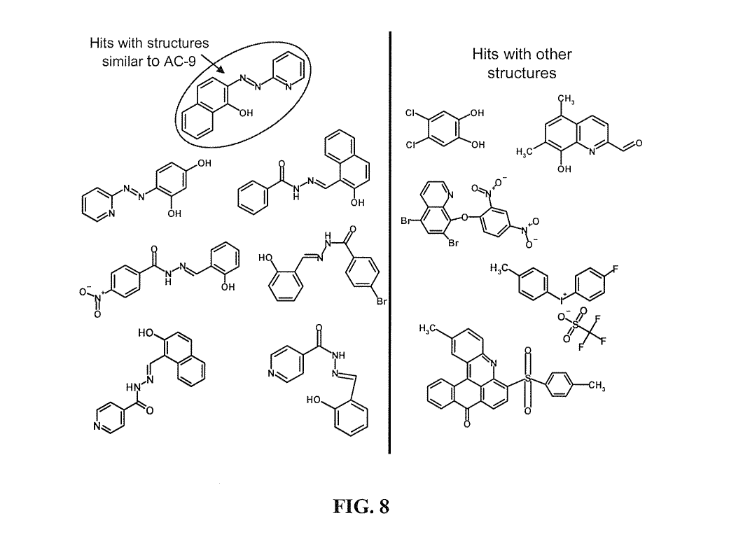

The structure of AC9 is given in FIG. 8, together with structures of the other 11 hits. There are 6 structures with similar attributes to the lead compound. Among this group, by first analysis, they possess a number of common structural features:
1. presence of an ionizable [pKa approximately 10] phenolic group;
2. the presence, in close proximity to the ionizable group of at least one heteroatom [N];
3. an azo [N—N] motif which bridges aryl groups; and
4. At least 2 substituted aryl groups with hydrophobic properties and the potential to
   engage in π-π and metal-π stacking interactions.

The compounds may chelate to metal ions through combination of the phenoxide ion and σ donor function of the nitrogen atoms. This leads to the formation of conformationally rigid chelated structures which then interact with the target. The degree of activity within the family is a function of these properties, complimented by pendant structural features (steric bulk, secondary co-ordination sites, hydrophobic pockets) which act in synergy.

AC9 was the only compound that showed killing activity in the presence of miconazole, and also the most active compound among the hits according to the XTT reduction inhibition checkerboard assay. The fact that AC9 had no activity on its own suggests that it has low toxicity in spite of the high concentration needed for anti-biofilm activity, which is 100-fold lower than the concentration of miconazole in Monistat®.

These experiments are inconsistent with a membrane-acting mechanism. Indeed, there was a clear difference between the behavior of the membrane-acting antiseptic chlorhexidine and miconazole (FIGS. 1B; 5). While increasing amounts of chlorhexidine ultimately eradicated persisters, these cells appeared completely invulnerable to miconazole, suggesting the presence of a saturable target. Synergistic action that was observed between miconazole and AC9 may have been a result of the simultaneous blocking of ergosterol synthesis, the secondary target(s) of miconazole, and the target(s) of AC9, resulting in effective killing of all C. albicans irrespective of the physiological state.

### Example 3

### Screening for Analogs of AC9

AC9 was used to perform a restricted structure search with the SciFinder Scholar® search engine (American Chemical Society, available, e.g., at https://scifinder.cas.org). The first parameter probed involved atomic substitution of all heteroatoms, which returned 264 unique structures, e.g., 1-(2-pyridylazo)-2-naphthol (shown below). In a second search, variation of the placement of the heteroatom in the pyridyl ring of AC-9 was permitted, and this search returned 444 unique structures, e.g., the chlorpyridyl substituted azo-naphthol (shown below). The screen is enlarged into a high-throughput screen format and is designed to lead to additional potentiator compounds.

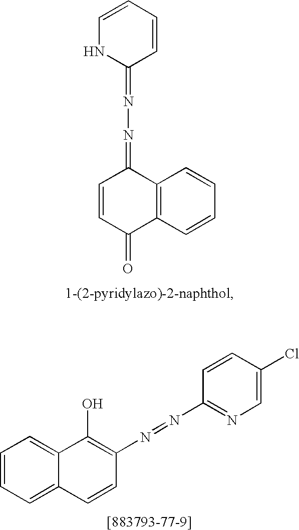

### Example 4

### In Vitro Validation of Miconazole Potentiators

Potentiators of miconazole such as AC9 are subjected to in vitro evaluation as discussed below to obtain candidate potentiators that can be advanced into animal toxicity and efficacy studies. These tests include activity (potency and spectrum), toxicity, and probability of resistance development.

### Activity

The initial screen and studies of AC9 described in Example 2 were performed with C. albicans strain CAF4-2. The activities of AC9 and other potentiators against a range of independent clinical isolates of C. albicans are determined.

The MIC90 of potentiators are measured (the minimal concentration in the presence of 100 μg/ml miconazole effective against 90% of tested strains). Biofilms are grown in microtiter plates (Ramage et al., Antimicrob. Agents Chemother. 45:2475-2479 (2001)) and challenged for 24 hrs with either 100 μg/ml miconazole, the potentiator at a concentration determined in a checkerboard as described for AC9, or a combination of the two. Under these conditions, AC9 completely eradicates biofilms of CAF4-2 in the presence of miconazole. A MIC90 that is similar to that found with the laboratory strain indicates that the potentiator can be advanced for further testing.

### Toxicity

Next, toxicity of potentiators against mammalian cells are determined. One of the intended applications of potentiators such as AC9 is a combination therapy with miconazole to treat relapsing vaginitis caused by C. albicans. One of the current treatments for relapsing vaginitis is local administration of a 2% miconazole ointment (Monistat). This is more than 6×10⁵ times higher than the MIC, and at this concentration miconazole could be lethal if administered systemically. The mouse LD₅₀ of miconazole is 519 mg/kg. This demonstrates tolerance of topical applications compared to systemic use. The “toxicity bar” for potentiators of miconazole are similarly higher compared to antiinfectives that are developed for systemic use. In vitro toxicity serves as one of the factors in prioritizing the leads obtained as miconazole potentiators.

In vitro cytotoxicity is monitored using three human cells lines: fibroblast IMR90, keratinocyte HEK001, and hepatocyte HepG2. Keratinocytes are cells of the epidermis and thus have high exposure to drugs being developed as topical agents. Fibroblasts, the main cells of connective tissue, are included in these assays because of their ubiquitous nature. Hepatocytes are included since they are a common site of drug toxicity.

Exponential cells are grown according to known conditions recommended by the ATCC and seeded at 10⁵ cells per well in a 96-well flat bottom plate (see Smee et al., J. Virol. Methods 106:71-79 (2002)). After overnight incubation to allow the cells to attach, media is removed and replaced with fresh media containing test compounds added at a two-fold serial dilution, similarly to performing an MIC assay. Cells are incubated overnight and cell viability is measured with the CellTiter-Glo Luminescent assay (Promega) according to the manufacturer's recommendations. This assay measures ATP production as an endpoint of cell viability and is proportional to cell number, with dead or damaged cells producing little or no ATP. The concentration of drug producing 50% cell viability (EC₅₀) is determined and used to calculate the therapeutic index, which for antiinfectives is EC₅₀/MIC. Since in the case of AC9 there is no MIC, 100 μg/ml is used as the minimal concentration at which AC9 potentiates complete killing of C. albicans by miconazole.

Membrane integrity, mitochondrial and lysosomal function are measured as additional endpoints of cytotoxicity using kits purchased from Xenometrix. Membrane integrity is measured as release of the cytosolic enzyme lactate dehydrogenase (LDH) into the cell medium. LDH is measured as the concurrent oxidation of NADH to NAD+ and reduction of lactate to pyruvate. Mitochondrial function is measured as reduction of XTT by mitochondria of metabolically active cells to formazan at 480 nm. To measure lysosomal activity, cells are incubated with neutral red (a dye preferentially absorbed into lysosomes of viable cells). Cells are destained and red color quantified at 540 nm (Waterfield et al., Arch. Toxicol. 72:588-596 (1998)). Assaying four parameters of cell viability in three cell types increases the likelihood of detecting toxicity if present.

Drugs are potentially directly toxic or converted to toxic metabolites by hepatic enzymes. Potentiators such as AC9 are tested for metabolic stability using cryopreserved hepatocytes. Intact hepatocytes contain all hepatic drug metabolizing enzymes, both microsomal and cytosolic as well as cofactors required for phase I oxidation and phase II conjugation. The assay is performed in 96-well plates with a porous membrane at the bottom. Intact hepatocytes are added to wells of containing compound in isotonic buffer. After 4 hrs of incubation, an equal volume of acetonitrile is added to stop the reaction and extract the test compound. Plates are centrifuged to filter the reaction through a porous membrane into a new 96-well recipient plate and samples are analyzed by LC-MS. Metabolic stability is expressed as a percentage of parent compound disappearance: 1-[parent compound concentration after incubation/parent compound concentration before incubation]×100 (Li et al., Chem. Biol. Interact. 121:17-35 (1999)).

Given that AC9 has no discernible activity against C. albicans when added alone, the parent compound is non-toxic against mammalian cells as well. Nonetheless a broader evaluation of cytotoxicity is undertaken, including: 1) assays with primary human cell culture systems that retain organ-specific properties; 2) genotoxicity studies; and 3) drug metabolism studies examining induction and inhibition of cytochrome P450 enzymes responsible for drug-drug interactions.

### Resistance Development

Next, the probability of resistance development to potentiators such as AC9 is evaluated. Miconazole is known to inhibit ergosterol biosynthesis. For topical applications, miconazole is used at a high concentration (2%). At this high concentration, miconazole has some additional action apart from inhibiting ergosterol biosynthesis. Resistance to this compound has not been a notable problem associated with Monistat treatment (http://www.rxmed.com/b.main/b2.pharmaceutical/b2.1.monographs/CPS-%20Monographs/CPS-%20(General %20Monographs-%20M)/MONISTAT.html). Biofilm cells treated with 100 μg/ml miconazole+200 μg/ml AC9 were completely eradicated (see FIG. 7), suggesting that resistance to AC9 may not occur with high probability.

To determine resistance probability, the probability of mutants that are able to survive (rather than to grow) in the presence of a potentiator such as AC9 and a high level of miconazole is determined. Mature biofilms are treated with a combination of 100 μg/ml miconazole+200 μg/ml of a potentiator such as AC9, incubated for 24 hrs (as recommended for MIC and MBC measurements), disrupted, washed and plated at 10⁹ CFU per plate, for a total of 20 plates. This allows the observation of low-probability resistance development (≦10⁻¹⁰). Any colonies that grow are then further examined in order to determine whether these are rare surviving persisters, or resistant mutants. Colonies are grown into biofilms, treated with 100 μg/ml miconazole+200 μg/ml of a potentiator such as AC9 as described above, and plated at around 100 cells per plate. No growth indicates the colonies are rare surviving persisters, whereas a significant number of colonies indicates the colonies are resistant mutants.

No resistance (<10⁻¹⁰) validates the compound, such as AC9, as a potentiator, and indicates that resistance development to the compound is due to a rare, and probably recessive mutation in the target gene; or, alternatively, that the compound hits more than one target and exhibits a non-specific mode of action. If a high probability of resistance development (>10⁻⁸) is observed, the compound is deprioritized. Finally, if a detectable, but reasonable rate of resistance development (10⁻¹⁰-10⁻⁸) is observed, further analysis is conducted aimed at learning whether resulting mutants are resistant to miconazole or to the potentiator using a checkerboard assay.

The checkerboard test is performed with a potentiator such as AC9 and miconazole using a planktonic culture of several independently isolated resistant clones (or biofilm, if resistance is only observed with biofilm cells). Sequential two-fold serial dilutions of miconazole are made along the x-axis of a microtiter plate and subsequent two-fold serial dilutions are made with a potentiator such as AC9 along the y-axis. Cells are added to each well containing combinations of miconazole and potentiator. Observing killing in the presence of an increase in the concentration of miconazole indicates that resistance of the mutant clone is due to miconazole. Similarly, observing killing in the presence of an increase in the concentration of the potentiator indicates that resistance is due to the potentiator.

If resistance is due to the potentiator such as AC9 for at least some cases, this indicates possible specificity of action and the identification of the resistant target is helpful in determining a detailed mechanism of action. Having a low, but measurable, level of resistance allows for advancing a lead into drug development, while at the same time providing a tool for determining the mechanism of action. Identifying the mutation in a resistant mutant is a standard approach to identify the mechanism of action for many currently-used antimicrobials (Lewis et al. (2002) Bacterial Resistance to Antimicrobials: Mechanisms, Genetics, Medical Practice and Public Health. New York: Marcel Dekker).

### Example 5

### Identifying Additional Miconazole Potentiators

Additional miconazole potentiators are identified using two approaches: identifying analogs of AC9 and performing a larger screen for additional, chemically unrelated potentiators.

### Analogs of Hit Compounds

Chemical analogs of AC9 (and of any additional leads identified from the larger screen, described below) are identified and tested for potentiation of biofilm eradication by miconazole, with the aim of obtaining compounds of superior activity. Searches are conducted using accessible MDL, PDB, and CML databases. Commercially available analogs are identified and tested for activity.

If no analogs are found that have superior activity to AC9 among the commercially available ones, an appropriate and representative (e.g., >30) number of analogs are synthesized utilizing SAR drivers (e.g., presence of hydrogen bonding atoms, location of sterically bulky groups, synergistic atom pairing combinations) and pharmacologic (ADMET) principles (including solubility, metabolic sites, ionizable sites, acidic/basic cavities, log P, metal binding sites) (see Xi et al., Chem. Biol. 9:925-931 (2002)). The synthesized analogs are tested for activity in the presence of miconazole against C. albicans biofilms and planktonic cells, and any synthesized analogs with superior activity to AC9 are validated as described in Example 3.

### Screening for Additional Lead Compounds

As described in Example 2, initially 5,000 compounds of the 16,000 from the ChemBridge library were screened for potentiators of miconazole action against C. albicans biofilms. A larger screen is conducted on the remaining 11,000 compounds from the ChemBridge library. Screening is performed as described in Example 2 using 96-well plates. Candida biofilms are formed by growing cells for 48 hrs at 37° C. in a shaking incubator in 96-well microtiter plates according to standard protocol (Ramage et al., Antimicrob. Agents Chemother. 45:2475-2479 (2001)). Eight wells of row one act as a negative control (cells alone), and AC9 are added to biofilms of row 12 at 10 μg/ml (positive control). 10 μg/ml miconazole is added to each well and 10 μg/ml of unique compounds is added to rows 2-11 and the plates are incubated for 24 hrs at 37° C. XTT is then added to each well at 1 mg/ml and the plates are incubated for an additional 4 hrs at 37° C. The amount of XTT converted to dye is quantified with a plate reader by subtracting the optical density at 690 nm from the optical density at 450 nm. The calculated OD value of the positive control (“hit”) according to our measurements is 0.4, and negative control (no inhibition) is >1. Positive hits are scored when the optical density of the experimental well is <0.7. If the hit rate of this screen is lower than that described in Example 2, or if no compounds are identified that kill biofilms in the presence of miconazole, the window is increased, and the additional hits are picked and tested.

An additional, larger screen is performed by the University of Wisconsin/Madison Small Molecule Screening Facility (http://www.hts.wisc.edu/) on a diverse library of 43,000 compounds, which includes the 16,000 ChemBridge DiverSet. These compounds come from a collection of NCl libraries (synthetic and natural compounds), Known Bioactives, which includes many compounds approved for use in humans, and ChemDiv. The compounds are tested for potentiating killing of biofilms in the presence of miconazole by the checkerboard method described in Example 2. Compounds that have the highest potentiating activity and the lowest (or no) direct activity are given priority. Analogs of these early leads are then identified as described above, and similarly tested for activity.

Subsequent validation of these leads is performed as described in Example 3. Validated leads are tested in animal studies in conjunction with medicinal chemistry optimization, and identified hits are advanced to clinical trials.

### EQUIVALENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of identifying a compound that potentiates the activity of an antifungal
agent, the method comprising:
contacting a fungus with an antifungal agent;
detecting the number of viable fungal cells in the presence of the antifungal agent;
contacting the fungus with a candidate potentiator compound;
detecting the number of viable fungal cells in the presence of the candidate potentiator compound; and
comparing the numbers of viable cells in the absence and presence of the candidate potentiator compound;
wherein a greater number of viable cells in the absence of the candidate potentiator compound than in the presence of the candidate potentiator compound is indicative that the candidate potentiator compound potentiates the activity of the antifungal agent.

2. The method of claim 1, further comprising contacting a second fungus with the candidate potentiator compound in the absence of the antifungal agent, and determining the number of viable cells of the second fungus in the absence and presence of the candidate potentiator compound, wherein the fungus and the second fungus are the same.

3. The method of claim 2, wherein the number of viable cells of the second fungus is substantially similar in the presence and absence of the candidate potentiator compound.

4. The method of claim 1, wherein the fungus is one or more of the following: Aspergillus flavus, Aspergillus fumigatus, Aspergillus glaucus, Aspergillus nidulans, Aspergillus niger, Aspergillus terreus, Blastomyces dermatitides, Candida albicans, Candida glabrata, Candida tropicalis, Candida parapsilosis, Candida krusei, Candida guillermondii, Coccidioides immitis, Cryptococcus neoformans, Cryptococcus albidus, Cryptococcus laurentii, Histoplasma capsulatum var. capsulatum, Histoplasma capsulatum var. duboisii, Paracoccidioides brasiliensis, Sporothrix schenckii, Absidia corymbifera, Rhizomucor pusillus, or Rhizopus arrhizus.

5. The method of claim 1, wherein the fungus comprises persister cells.

6. A method of inhibiting the growth of, or killing, a fungus, the method comprising contacting the fungus with (i) an antifungal agent, and (ii) one or more potentiator compounds of Formulae I, II, III, and IV, or pharmaceutically acceptable salts, hydrates, and solvates thereof: 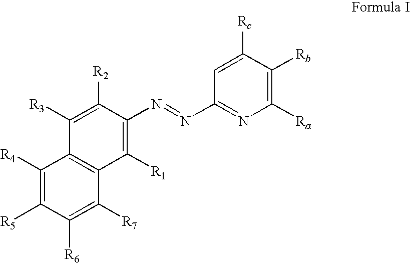
wherein R₁-R₇ and Rₐ-R_{c} are each independently —H, halogen, amino, alkylamino,
nitro, hydroxyl, cyano, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃ alkyl, —NHC(O)—C₁-C₆ alkyl, aryl, arylalkyl, heteroaryl, heteroarylalkyl; 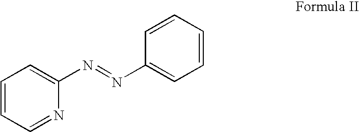
wherein any one or more —H can be independently substituted with any one of the
following substituents: halogen; —NO₂; —NH₂; alkylamino, hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; 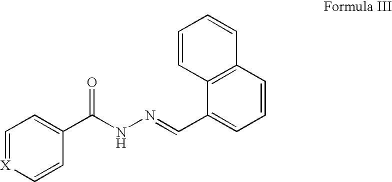
wherein any one or more —H can be independently substituted with any one of the
following substituents: halogen; —NO₂; —NH₂; alkylamino, hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; and wherein X is N or C(H); and 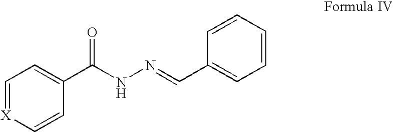
wherein any one or more —H can be independently substituted with any one of the
following substituents: halogen; —NO₂; —NH₂; alkylamino, hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; and wherein X is N or C(H);
thereby inhibiting the growth of, or killing, the fungus.

7. The method of claim 6, wherein the potentiator compound is of Formula I.

8. The method of claim 6, wherein the potentiator compound is of Formula II.

9. The method of claim 6, wherein the potentiator compound is of Formula III.

10. The method of claim 6, wherein the potentiator compound is of Formula IV.

11. The method of claim 6, wherein the potentiator compound potentiates the activity of the antifungal agent.

12. The method of claim 6, wherein the potentiator compound is not an antifungal compound.

13. The method of claim 6, wherein the fungus is one or more of the following: Aspergillus flavus, Aspergillus fumigatus, Aspergillus glaucus, Aspergillus nidulans, Aspergillus niger, Aspergillus terreus, Blastomyces dermatitides, Candida albicans, Candida glabrata, Candida tropicalis, Candida parapsilosis, Candida krusei, Candida guillermondii, Coccidioides immitis, Cryptococcus neoformans, Cryptococcus albidus, Cryptococcus laurentii, Histoplasma capsulatum var. capsulatum, Histoplasma capsulatum var. duboisii, Paracoccidioides brasiliensis, Sporothrix schenckii, Absidia corymbifera, Rhizomucor pusillus, or Rhizopus arrhizus.

14. The method of claim 6, wherein the antifungal agent is Amphotericin B, miconazole, clotrimazole, fluconazole, itraconazole, ketoconazole, ravuconazole, posaconazole, voriconazole, caspofungin, micafungin, FK463, anidulafungin (LY303366), hydroxystilbamidine, 5-fluorocytosine, flucytosine, iodide, terbinafine, Nystatin, griseofulvin, or ciclopirox.

15. A method of treating a fungal infection in a subject in need thereof, the method comprising administering to the subject an effective amount of an antifungal agent in combination with an effective amount of one or more potentiator compounds of Formulae I, II, III, and IV, or pharmaceutically acceptable sales, hydrates, and solvates thereof: 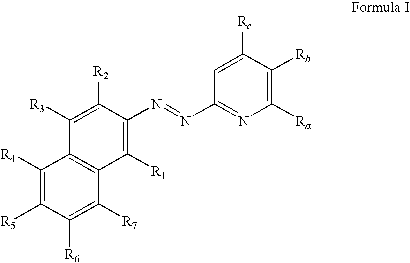
wherein R₁-R₇ and Rₐ-R_{c} are each independently —H, halogen, amino, alkylamino,
nitro, hydroxyl, cyano, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃ alkyl, —NHC(O)—C₁-C₆ alkyl, aryl, arylalkyl, heteroaryl, heteroarylalkyl; 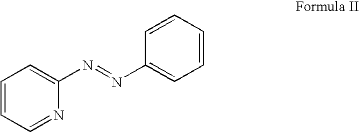
wherein any one or more —H can be independently substituted with any one of the
following substituents: halogen; —NO₂; —NH₂; alkylamino, hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; 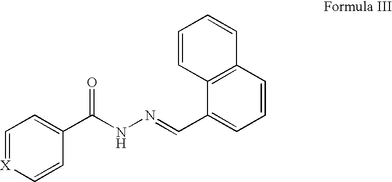
wherein any one or more —H can be independently substituted with any one of the
following substituents: halogen; —NO₂; —NH₂; alkylamino, hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; and wherein X is N or C(H); and 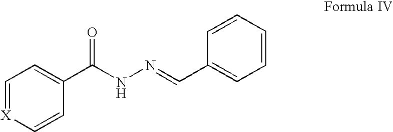
wherein any one or more —H can be independently substituted with any one of the
following substituents: halogen; —NO₂; —NH₂; alkylamino, hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; and wherein X is N or C(H);
thereby treating the fungal infection.

16. The method of claim 15, wherein the potentiator compound is of Formula I.

17. The method of claim 15, wherein the potentiator compound is of Formula II.

18. The method of claim 15, wherein the potentiator compound is of Formula III.

19. The method of claim 15, wherein the potentiator compound is of Formula IV.

20. The method of claim 15, wherein the potentiator compound potentiates the activity of the antifungal agent.

21. The method of claim 15, wherein the potentiator compound is not an antifungal compound.

22. The method of claim 15, wherein the fungal infection comprises one or more of the following: Aspergillus flavus, Aspergillus fumigatus, Aspergillus glaucus, Aspergillus nidulans, Aspergillus niger, Aspergillus terreus, Blastomyces dermatitidis, Candida albicans, Candida glabrata, Candida tropicalis, Candida parapsilosis, Candida krusei, Candida guillermondii, Coccidioides immitis, Cryptococcus neoformans, Cryptococcus albidus, Cryptococcus laurentii, Histoplasma capsulatum var. capsulatum, Histoplasma capsulatum var. duboisii, Paracoccidioides brasiliensis, Sporothrix schenckii, Absidia corymbifera, Rhizomucor pusillus, or Rhizopus arrhizus.

23. The method of claim 15, wherein the antifungal agent is Amphotericin B, miconazole, clotrimazole, fluconazole, itraconazole, ketoconazole, ravuconazole, posaconazole, voriconazole, caspofungin, micafungin, FK463, anidulafungin (LY303366), hydroxystilbamidine, 5-fluorocytosine, flucytosine, iodide, terbinafine, Nystatin, griseofulvin, or ciclopirox.

24. The method of claim 15, wherein the fungal infection is aspergillosis, blastomycosis, candidiasis, coccidioidomycosis, cryptococcosis, histoplasmosis, paracoccidiomycosis, sporotrichosis, or zygomycosis.

25. The method of claim 15, wherein the fungal infection is associated with a catheter, an orthopedic prostheses, or a heart valve.

26. A method of inhibiting the growth of, or killing, a fungus, the method comprising contacting the fungus with effective amounts of (i) an antifungal agent, and (ii) one or more of potentiator Compounds 1-12: 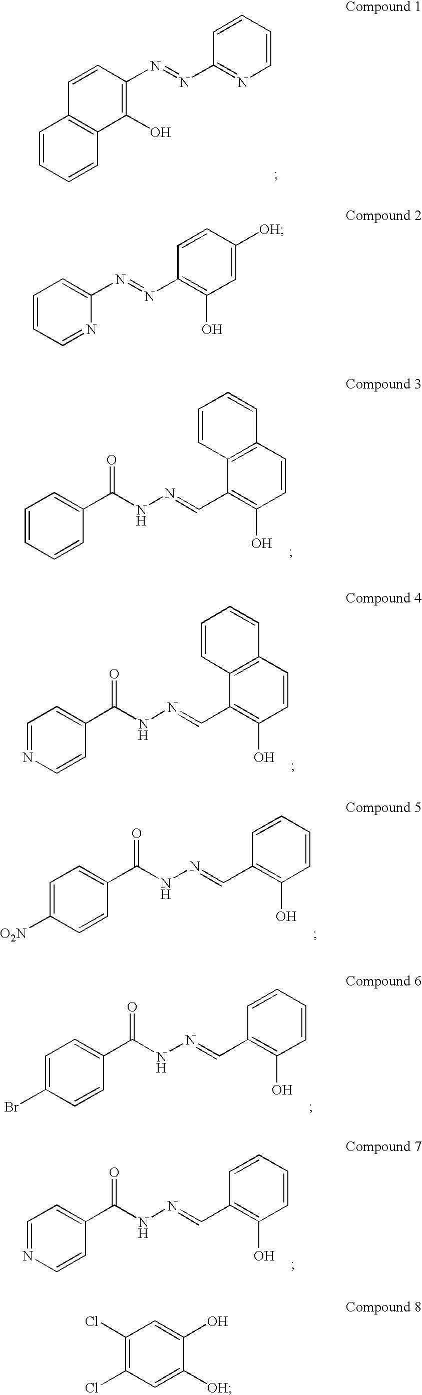 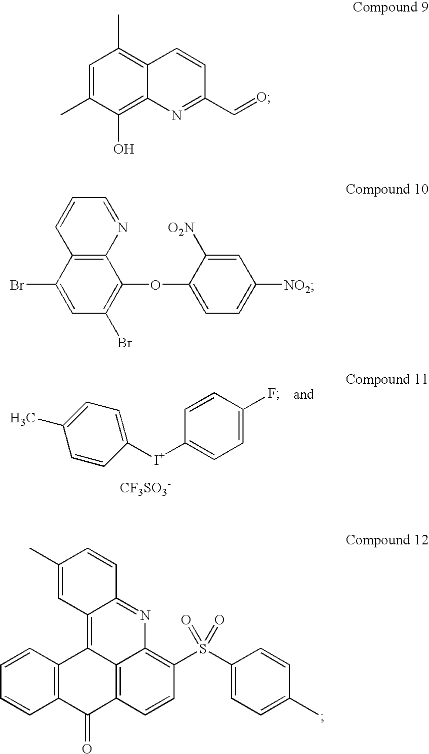
or one or more of a pharmaceutically acceptable salt, hydrate, or solvate of potentiator Compounds 1-12, thereby inhibiting the growth of, or killing, the fungus.

27. The method of claim 26, wherein the potentiator compound potentiates the activity of the antifungal agent.

28. The method of claim 26, wherein the potentiator compound is not an antifungal compounds.

29. The method of claim 26, wherein the fungus is one or more of the following: Aspergillus flavus, Aspergillus fumigatus, Aspergillus glaucus, Aspergillus nidulans, Aspergillus niger, Aspergillus terreus, Blastomyces dermatitidis, Candida albicans, Candida glabrata, Candida tropicalis, Candida parapsilosis, Candida krusei, Candida guillermondii, Coccidioides immitis, Cryptococcus neoformans, Cryptococcus albidus, Cryptococcus laurentii, Histoplasma capsulatum var. capsulatum, Histoplasma capsulatum var. duboisii, Paracoccidioides brasiliensis, Sporothrix schenckii, Absidia corymbifera, Rhizomucor pusillus, or Rhizopus arrhizus.

30. The method of claim 26, wherein the antifungal agent is Amphotericin B, miconazole, clotrimazole, fluconazole, itraconazole, ketoconazole, ravuconazole, posaconazole, voriconazole, caspofungin, micafungin, FK463, anidulafungin (LY303366), hydroxystilbamidine, 5-fluorocytosine, flucytosine, iodide, terbinafine, Nystatin, griseofulvin, or ciclopirox.

31. A method of treating a fungal infection in a subject in need thereof, the method comprising administering to the subject an effective amount of an antifungal agent in combination with an effective amount of one or more of potentiator Compounds 1-12: 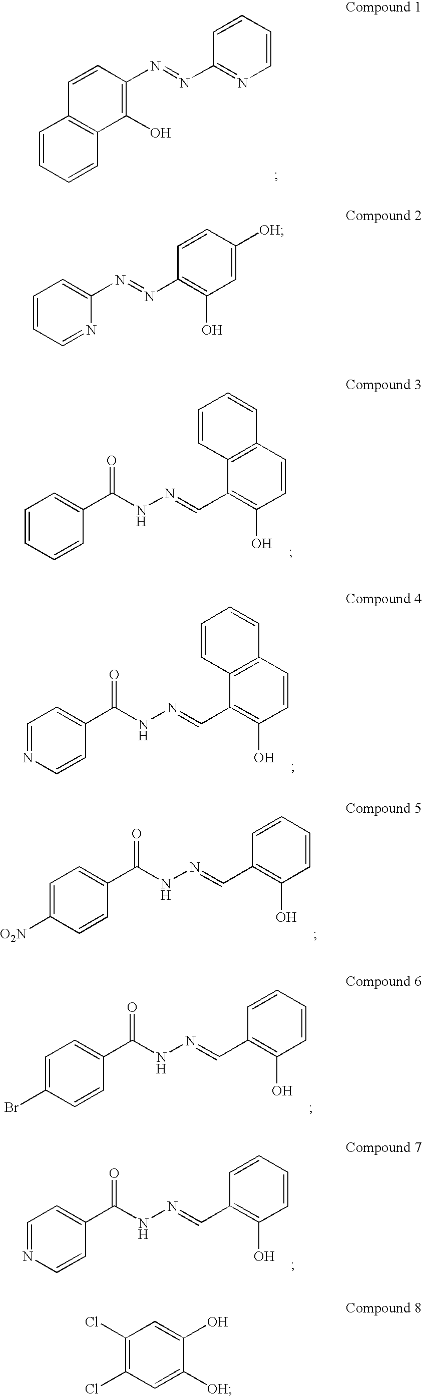 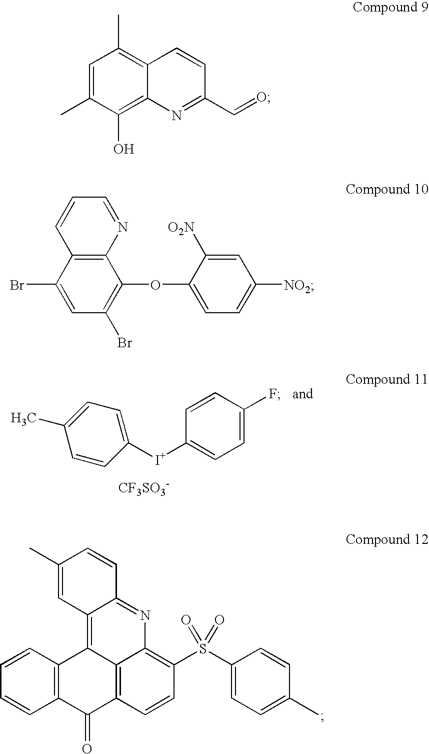
or one or more pharmaceutically acceptable salts, hydrates, or solvates of potentiator Compounds 1-12, thereby treating the fungal infection.

32. The method of claim 31, wherein the potentiator compound potentiates the activity of the antifungal agent.

33. The method of claim 31, wherein the compound is not an antifungal compound.

34. The method of claim 31, wherein the fungal infection is due to one or more of the following: Aspergillus flavus, Aspergillus fumigatus, Aspergillus glaucus, Aspergillus nidulans, Aspergillus niger, Aspergillus terreus, Blastomyces dermatitidis, Candida albicans, Candida glabrata, Candida tropicalis, Candida parapsilosis, Candida krusei, Candida guillermondii, Coccidioides immitis, Cryptococcus neoformans, Cryptococcus albidus, Cryptococcus laurentii, Histoplasma capsulatum var. capsulatum, Histoplasma capsulatum var. duboisii, Paracoccidioides brasiliensis, Sporothrix schenckii, Absidia corymbifera, Rhizomucor pusillus, or Rhizopus arrhizus.

35. The method of claim 31, wherein the antifungal agent is Amphotericin B, miconazole, clotrimazole, fluconazole, itraconazole, ketoconazole, ravuconazole, posaconazole, voriconazole, caspofungin, micafungin, FK463, anidulafungin (LY303366), hydroxystilbamidine, 5-fluorocytosine, flucytosine, iodide, terbinafine, Nystatin, griseofulvin, or ciclopirox.

36. The method of claim 31, wherein the fungal infection is aspergillosis, blastomycosis, candidiasis, coccidioidomycosis, cryptococcosis, histoplasmosis, paracoccidiomycosis, sporotrichosis, or zygomycosis.

37. The method of claim 31, wherein the fungal infection is associated with a catheter, an orthopedic prostheses, or a heart valve.

38. A method of inhibiting the growth of, or killing, a fungus, the method comprising contacting the fungus with effective amounts of (i) an antifungal agent, and (ii) potentiator Compound 1: 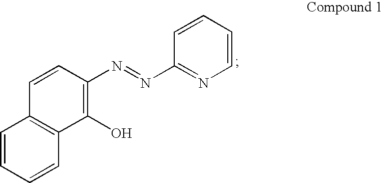
or a pharmaceutically acceptable salt, hydrate, or solvate of potentiator Compound 1, thereby inhibiting the growth of, or killing, the fungus.

39. The method of claim 38, wherein the potentiator compound potentiates the activity of the antifungal agent.

40. A method of treating a fungal infection in a subject in need thereof, the method comprising administering to the subject an effective amount of an antifungal agent in combination with an effective amount of one or more of potentiator Compound 1: 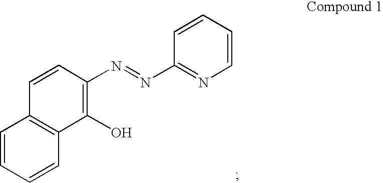
or a pharmaceutically acceptable salt, hydrate, or solvate of potentiator Compound 1, thereby treating the fungal infection.

41. The method of claim 40, wherein the potentiator compound potentiates the activity of the antifungal agent.

42. A method of treating relapsing vaginitis in a subject in need thereof, the method comprising administering to the subject an effective amount of miconazole in combination with an effective amount of potentiator Compound 1: 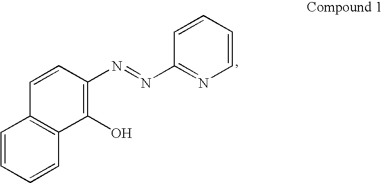
thereby treating the relapsing vaginitis in the subject.

43. The method of claim 42, wherein the relapsing vaginitis is caused by a Candida albicans infection.

44. The method of claim 42, wherein the relapsing vaginitis is caused by Candida albicans persister cells.
